(19) United States
(12) Patent Application Publication
(10) Pub. No.: US 2009/0155900 A1
(43) Pub. Date: Jun. 18, 2009

(54) CELL CULTURE MATRICES

(75) Inventors: Mohanachari Vemuri, Grand Island, NY (US); Sackamone Phommachanh, Buffalo, NY (US); Katherine Wagner, Kenmore, NY (US); Mary Tilkins, North Tonawanda, NY (US)

Correspondence Address: INVITROGEN CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/333,780

(22) Filed: Dec. 12, 2008

Related U.S. Application Data
(60) Provisional application No. 61/013,833, filed on Dec. 14, 2007.

(51) Int. Cl. C12N 5/08; C12N 5/02; C12N 5/00

(52) U.S. Cl. 435/366; 435/402; 435/407

(57) ABSTRACT

The present invention relates to cell culture, more specifically to cell culture which may be cell culture such as stem cell culture, embryonic stem cell (ESC) culture and primary cell culture. Disclosed herein are compositions of matter, including without limitation cell matrices, matrix-forming formulations and cell cultures, wherein the cells may be cells such as stem cells, such as ESC, or primary cells, such as keratinocyte and fibroblast cells. Also disclosed herein are articles of manufacture comprising one or more of the compositions of matter of the invention, methods of making and using the compositions of matter and articles of manufacture of the invention, business methods, and tangible media comprising instructions or plans for one or more of the methods and compositions of the invention.

### CROSS REFERENCE TO RELATED APPLICATIONS

This application claims the benefit of U.S. Provisional Application No. 61/013,833, filed Dec. 14, 2007, the entire disclosure of which is incorporated herein by reference.

### FIELD OF THE INVENTION

The present invention concerns compositions and methods relating to cell culture.

### BACKGROUND OF THE INVENTION

All publications, patent applications, and patents mentioned in this specification are herein incorporated by reference to the same extent as if each individual publication, patent application, or patent was specifically and individually indicated to be incorporated by reference.

Many cells, especially cells of metazoan organisms, exhibit altered characteristics when placed in contact with other cells and/or biological materials. As an example, when the surface of the skin is cut, cells in the epidermis will divide to close and “heal” the “wound”. Once the trauma site has been closed off, cells typically stop dividing. This effect is referred to as contact inhibition. In many forms of cancer, cells lose their contact inhibition and inappropriately divide. Contacts between cells may also result in cell signaling, both by direct contact between cells and through soluble factors. Some cells exhibit an enhanced ability to divide and/or differentiate when in contact (e.g., physical contact) with other cells and/or biological materials (e.g., cell-free matrices).

A stem cell is a unique type of cell having many potential uses. Three broad categories of mammalian stem cells are known: embryonic stem cells, adult stem cells and cord blood stem cells.

Embryonic stem cells (ES cells, ESCs) are derived from the epiblast tissue of the inner cell mass (ICM) of an early stage embryo such as a blastocyst or an earlier morula stage embryo. This method involves more advanced-stage embryos and uses a process that destroys the embryo.

Another method uses a cell derived from an early stage embryo during pre-implantation genetic diagnosis (PGD). In PGD, a single cell is plucked from an embryo when it is a three-day-old ball of only eight cells, and tested for defects such as cystic fibrosis. More than 2,000 babies have been born worldwide following PGD. A single cell removed from an embryo can be grown into many cells overnight, and some of those can then be turned into ESC (Klimanskaya et al., Nature 444:481-485, 2006).

The properties and useful characteristics of ESC and other stem cells may vary according to the method of their preparation. Studies comparing ESC prepared by these and other methods are ongoing, and new methods of preparing ESC will arise in time. One skilled in the art will be able to practice the invention using cells, including stem cells, including ES cells, of any suitable type.

ESCs are pluripotent, giving rise during development to all derivatives of the three primary germ layers: ectoderm, endoderm and mesoderm (Pal et al., Regen. Med. 2:179-192, 2007). Ideally, they can develop into each of the more than 200 cell types of the adult body if properly stimulated.

Human embryonic stem (hES) cells are known in the art and can be prepared in various manners, including as non-limiting examples those described by Thomson et al. (U.S. Pat. No. 5,843,780; Science 282:1145-1147, 1998) and Embryonic Stem Cells (Methods in Molecular Biology Vol. 135, Turkson, Ed., Human Press, Totawa, N.J., 2002).

A significant limitation to the use of stem cells is that they are traditionally cultured on a layer of feeder cells to provide a surface for the stem cells to attach to and various (uncharacterized) soluble factors that prevent differentiation. Culture of human embryonic stem cells (hESCs) was initially dependent on the use of mouse cells (MEFs) as a feeder layer. The use of feeder layers raises several concerns: the potential presence of xenogeneic contaminants which might, for example, restrict transplantation of hES cells to humans; the potential for variability in MEFs from batch-to-batch and laboratory-to-laboratory, which may contribute to variability in experimental results; and the use of a second population of cells (the feeder cells), which increases the workload and subsequently limits the large-scale culture of human ES cells.

Accordingly, a goal in stem cell technology has been the development of feeder-free cultures. One approach has been to develop biological extracts which, although undefined, do not comprise living cells but do provide molecular components necessary for stem cell (e.g., ES cell) undifferentiated growth. Over time, researchers have come to understand that these necessary components include, in addition to culture media, (i) a matrix for stem cell attachment and (ii) various uncharacterized soluble factors that promote stem cell growth but suppress, or at least do not induce, differentiation.

One commonly used matrix is Matrigel™, which is an extracellular matrix extracted from murine Engelbreth-Holm-Swarm tumor cells (Kleinman et al., Biochemistry: 21: 6188-6193, 1982). Soluble factors are often provided by the use of conditioned media (i.e., media that has been in contact with feeder cells that release soluble factors into the media).

Although it has been characterized to some degree, Matrigel™ is not a completely defined formulation. That is, the identities of all of substances present within Matrigel™ are not known, much less quantified.

The use of undefined formulations in cell cultures, although often done, is less than ideal. Undefined formulations are subject to lot-to-lot variation, in terms of their composition, during production. Such variation can be minimized but not eliminated, which has the consequences of increased workload (testing and/or characterizing the undefined formulation before its use or after its failure, time spent repeating experiments, etc.) and reduced accuracy and precision in experimental results. Defined stem cell culture conditions, with defined stem cell matrices and defined culture media, are ultimately preferable.

Another related goal in stem cell technology is the development of xeno-free (i.e., free of substances from any animal other than the species of animal from which the stem cells are derived) culture conditions. Serum-free culture conditions are also sought, as serum is a mixture of many components. Although it is relatively well-characterized, sera can vary from lot to lot, and human sera, needed for xeno-free growth of human ESCs, are more expensive than non-human sera. For example, the commonly used fetal bovine serum (FBS) cannot by definition be used to prepare xeno-free human cell cultures.

One tool that would be helpful in achieving such goals would be a simple, defined cell (e.g., stem cell) matrix that has useful characteristics that approximate, preferably improve upon and/or increase the number of, the useful characteristics of Matrigel™, the current “gold standard” of hESC culture matrices. In conjunction with certain media, such a matrix could be used, for example, to generate stem cell cultures that are defined, serum-free and/or xeno-free. Of course, such matrices may also be used with other cell types as well. For example, non-stem cells which exhibit enhanced growth characteristics when in contact with a matrix.

A variety of stem cells and cell lines can be used to practice the invention. For example, the stem cells can be embryonic stem cells (ES cells, or ESC), such as human embryonic stem cells (hESC) or non-human primate ES cells. Exemplary human embryonic stem cells include without limitation BG01, BG02, BG03, BG01v, CHA-hES-1, CHA-hES-2, FCNCBS1, FCNCBS2, FCNCBS3, H1, H7, H9, H13, H14, HSF-1, H9.1, H9.2, HES-1, HES-2, HES-3, HES-4, HES-5, HES-6, hES-1-2, hES-3-0, hES-4-0, hES-5-1, hES-8-1, hES-8-2, hES-9-1, hES-9-2, hES-101, hICM8, hICM9, hICM40, hICM41, hICM42, hICM43, HSF-6, HUES-1, HUES-2, HUES-3, HUES-4 HUES-5, HUES-6, HUES-7 HUES-8, HUES-9, HUES-10, HUES-11, HUES-12, HUES-13, HUES-14, HUESS-15, HUES-16, HUES-17, 13, 14, 16, 13.2, 13.3, 16.2, J3, J3.2, MB01, MB02, MB03, Miz-hES1, RCM-1, RLS ES 05, RLS ES 07, RLS ES 10, RLS ES 13, RLS ES 15, RLS ES 20, RLS ES 21, SA01, SA02, and SA03.

Stem cells and ES cells from non-human primates include those from the rhesus monkey, Macaca mulatta (Byrne et al., Nature 450: 497-502, 2007), and the common marmoset, Callithrix jacchus (Thompson and Marshall, Curr. Top. Dev. Biol. 38:133-165, 1998). Other non-human primates include without limitation Allenopithecus nigroviridis (Allen's swamp monkey), Allocebus trichotis (hairy-eared mouse lemur), Alouatta sp. (howler monkey), Alouatta seniculus (red howler), Aotus sp. (owl monkey), Arctocebus sp. (angwantibo), Ateles sp. (spider monkey), Ateles paniscus (black spider monkey), Avahi sp. (woolly indri), Brachyteles sp. (muriqui), Cacajao sp. (uakari), Callicebus sp. (titi), Callicebus moloch (dusky titi), Callimico goeldii (Goeldi's marmoset), Callithrix sp. (marmoset), Callithrix jacchus (common marmoset), Callithrix pygmaea (pygmy marmoset), Cebus sp. (capuchin monkey), Cebus apella (tufted capuchin), Cercocebus sp. (mangabey), Cercocebus atys (sooty mangabey), Cercopithecus sp. (guenon), Chemogaleus sp. (dwarf lemur), Chiropotes sp. (bearded saki), Chlorocebus sp. (vervet), Colobus sp. (black-and-white colobus), Daubentonia madagascariensis (aye-aye), Erythrocebus patas (patas monkey), Eulemur sp. (brown lemur), Euoticus sp. (needle-clawed bushbaby), Galago sp. (lesser bushbaby), Gorilla sp. (gorilla). Hapalemur sp. (bamboo lemur), Hoolock sp. (hoolock gibbon), Hylobates sp. (gibbon), Indri indri (indri), Lagothrix sp. (woolly monkey), Lemur catta (ring-tailed lemur), Leontopithecus sp. (lion tamarin), Leontopithecus chrysomelas (golden-headed lion tamarin), Leontopithecus rosalia (golden lion tamarin), Lepilemur sp. (sportive lemur), Lophocebus sp. (crested mangabey), Loris sp. (slender loris), Macaca sp. (macaque), Macaca arctoides (stump-tailed macaque), Macaca fascicularis (long-tailed macaque), Macaca fuscata (Japanese macaque), Macaca mulatta (rhesus macaque), Macaca nemestrina (pigtail macaque), Macaca nigra (crested black macaque), Mandrillus sp. (drill), Microcebus sp. (mouse lemur), Miopithecus sp. (talapoin), Mirza sp. (giant mouse lemur), Nasalis larvatus (proboscis monkey), Nomascus sp. (crested gibbon), Nomascus leucogenys (white-cheeked gibbon), Nycticebus sp. (slow loris), Oreonax flavicauda (yellow-tailed woolly monkey), Otolemur sp. (greater galago), Pan paniscus (bonobo), Pan troglodytes (chimpanzee), Papio sp. (baboon), Papio anubis (olive baboon), Papio cynocephalus (yellow baboon), Perodicticus potto (potto), Phaner sp. (fork-marked lemur), Piliocolobus sp. (red colobus), Pithecia sp. (saki monkey), Pongo sp. (orangutan), Presbytis sp. (surili), Procolobus verus (olive colobus), Prolemur simus (greater bamboo lemur), Propithecus sp. (sifaka), Propithecus diadema (diademed sifaka), Pygathrix sp. (douc), Rhinopithecus sp. (snub-nosed monkey), Rhinopithecus roxellana (golden snub-nosed monkey), Saguinus sp. (tamarin), Saguinus oedipus (cotton-top tamarin), Saimiri sp. (squirrel monkey), Semnopithecus sp. (gray langur), Simias concolor (pig-tailed langur), Symphalangus syndactylus (siamang), Tarsius sp. (tarsier), Theropithecus gelada (gelada baboon), Trachypithecus sp. (purple-faced langur) and Varecia sp. (ruffed lemur).

Stem cells and ES cells can also be prepared from other animal species, including without limitation ungulates (cows, sheep, goats, pigs, etc.), horses, dogs and cats (for reviews, see Tecirlioglu and Trounson, Reprod. Fertil. Dev. 19:740-747, 2007, and Keefer et al., Anim Reprod Sci. 98:147-168, 2007). Specific canine ES cells have been described by Hayes et al., Stem Cells, 26: 565-473, 2008.

### SUMMARY OF THE INVENTION

### Introduction

The invention is directed, in part, to compositions and methods for cultivating and maintaining cells (e.g., eukaryotic cells). In specific aspects, the invention provides, in part, for example, a composition (e.g., a matrix) which alter one or more cellular characteristic of a cell placed in close proximity (e.g., in direct contact, within from about one to about thirty, from about one to about ten, from about one to about five, from about three to about thirty, from about three to about ten, from about five to about thirty, etc. cell widths) or in the same environmental region (e.g., in the same culture vessel or organism).

Aspects of the invention described below include without limitation compositions of matter; articles of manufacture; kits comprising the compositions of matter and/or articles of manufacture of the invention; methods of making the compositions of matter and articles of manufacture of the invention; methods of using the compositions of matter and articles of manufacture of the invention; business methods relating to methods or compositions described herein; and objects comprising instructions, written in tangible form, for making or using one or more of the compositions of matter or articles of manufacture of the invention.

Various commercial embodiments of the invention use the tradename CELLstart™. The tradename is used generically throughout herein to refer to any cell matrix of the invention (e.g., stem cell matrices); however, specific CELLstart™ products are described herein, including without limitation the 1× and 10× formulations described in Example 1.

Compositions of Matter

In one aspect, the invention provides, in part, compositions of matter.

Embodiments of this aspect of the invention include, without limitation, cell matrices (e.g., stem cell matrices), cell cultures (e.g., stem cell matrices), matrix-forming formulations and other formulations disclosed herein.

The term “formulation,” as used herein, means any assembled composition of matter that comprises two or more different molecular components, excluding water. Such components can be present in any detectable amount greater than 0 and in any state of matter (e.g., liquid, solid, gas, ions) or mixture (e.g., dissolved, suspended, vaporized, etc.). Unlike an extract, which is a mixture derived from a biological source, a formulation is synthetic, and is assembled component-by-component using pure compounds.

Matrices

In a first embodiment, the invention provides, in part, cell matrices, which may be cell matrices such as primary cell matrices or stem cell matrices, such as ESC matrices, wherein said cell matrices:

(a) comprise, consist of, or consist essentially of Fibronectin and Albumin, and/or functional derivatives of such proteins;

(b) comprise, consist of, or consist essentially of amounts of Fibronectin and Albumin sufficient for the formation of a cellular matrix; and

(c) support the growth of stem cells in the presence of culture media. Specific embodiments of the invention may contain only one or two of (a), (b), or (c) above.

Other compositions of matter disclosed herein include without limitation cell cultures, which may be cell cultures such as primary cell cultures or stem cell cultures, such as ESC cultures, comprising, consisting of, or consisting essentially of the cell matrices of the invention; formulations for forming the cell matrices, which may be cell matrices such as primary cell matrices or stem cell matrices, such as ESC matrices, of the invention (matrix-coating formulations); and other formulations.

Fibronectin

As used herein, the term “Fibronectin” is meant to encompass any group of fibronectin molecules (proteins) that can be used to form cell matrices (e.g., stem cell matrices) of the invention. In many instances, this means that the Fibronectin employed will have functional charcteristics which allow for the formation of matrices having properties described herein. This includes proteins which have one or more functional domains of Fibronectin and proteins with sufficient sequence homology such that they contain one or more functional activity which allows for these proteins to participate in the function of matrices of the invention.

Thus, the term “Fibronectin” encompasses naturally occurring isoforms, such as those that occur in different tissues in the body. These include without limitation plasma fibronectin, cellular fibronectin and fetal fibronectin.

A Fibronectin for use in the invention may be purified from any selected species, including without limitation human (Homo sapiens) and non-human primates. Non-human primates include without limitation Allenopithecus nigroviridis (Allen's swamp monkey), Allocebus trichotis (hairy-eared mouse lemur), Alouatta sp. (howler monkey), Alouatta seniculus (red howler), Aotus sp. (owl monkey), Arctocebus sp. (angwantibo), Ateles sp. (spider monkey), Ateles paniscus (black spider monkey), Avahi sp. (woolly indri), Brachyteles sp. (muriqui), Cacajao sp. (uakari), Callicebus sp. (titi), Callicebus moloch (dusky titi), Callimico goeldii (Goeldi's marmoset), Callithrix sp. (marmoset), Callithrix jacchus (common marmoset), Callithrix pygmaea (pygmy marmoset), Cebus sp. (capuchin monkey), Cebus apella (tufted capuchin), Cercocebus sp. (mangabey), Cercocebus atys (sooty mangabey), Cercopithecus sp. (guenon), Chemogaleus sp. (dwarf lemur), Chiropotes sp. (bearded saki), Chlorocebus sp. (vervet), Colobus sp. (black-and-white colobus), Daubentonia madagascariensis (aye-aye), Erythrocebus patas (patas monkey), Eulemur sp. (brown lemur), Euoticus sp. (needle-clawed bushbaby), Galago sp. (lesser bushbaby), Gorilla sp. (gorilla). Hapalemur sp. (bamboo lemur), Hoolock sp. (hoolock gibbon), Hylobates sp. (gibbon), Indri indri (indri), Lagothrix sp. (woolly monkey), Lemur catta (ring-tailed lemur), Leontopithecus sp. (lion tamarin), Leontopithecus chrysomelas (golden-headed lion tamarin), Leontopithecus rosalia (golden lion tamarin), Lepilemur sp. (sportive lemur), Lophocebus sp. (crested mangabey), Loris sp. (slender loris), Macaca sp. (macaque), Macaca arctoides (stump-tailed macaque), Macaca fascicularis (long-tailed macaque), Macaca fuscata (Japanese macaque), Macaca mulatta (rhesus macaque), Macaca nemestrina (pigtail macaque), Macaca nigra (crested black macaque), Mandrillus sp. (drill), Microcebus sp. (mouse lemur), Miopithecus sp. (talapoin), Mirza sp. (giant mouse lemur), Nasalis larvatus (proboscis monkey), Nomascus sp. (crested gibbon), Nomascus leucogenys (white-cheeked gibbon), Nycticebus sp. (slow loris), Oreonax flavicauda (yellow-tailed woolly monkey), Otolemur sp. (greater galago), Pan paniscus (bonobo), Pan troglodytes (chimpanzee), Papio sp. (baboon), Papio anubis (olive baboon), Papio cynocephalus (yellow baboon), Perodicticus potto (potto), Phaner sp. (fork-marked lemur), Piliocolobus sp. (red colobus), Pithecia sp. (saki monkey), Pongo sp. (orangutan), Presbytis sp. (surili), Procolobus verus (olive colobus), Prolemur simus (greater bamboo lemur), Propithecus sp. (sifaka), Propithecus diadema (diademed sifaka), Pygathrix sp. (douc), Rhinopithecus sp. (snub-nosed monkey), Rhinopithecus roxellana (golden snub-nosed monkey), Saguinus sp. (tamarin), Saguinus oedipus (cotton-top tamarin), Saimiri sp. (squirrel monkey), Semnopithecus sp. (gray langur), Simias concolor (pig-tailed langur), Symphalangus syndactylus (siamang), Tarsius sp. (tarsier), Theropithecus gelada (gelada baboon), Trachypithecus sp. (purple-faced langur) and Varecia sp. (ruffed lemur).

The term “Fibronectin” also encompasses synthetic molecules, such as synthetic peptides derived from full-length fibronectin proteins, fibronectin molecules produced by recombinant DNA technology, and the like.

Synthetic peptides, comprising from 10 to about 200 amino acids can be synthesized in vitro according to methods known in the art. After amino acids are chemically polymerized in a sequence derived from the full peptide sequence of Fibronectin, synthetic peptides are purified and/or chemically modified (e.g., glycosylation, cross-linking). Fibronectin can be produced using recombinant DNA technology. A gene for a Fibronectin protein is cloned directly or stepwise into an expression vector. The Fibronectin gene is induced (stimulated to express), resulting in increased synthesis of the Fibronectin protein. The Fibronectin may be part of a fusion protein.

Albumin

As used herein, the term “Albumin” is meant to encompass any group of albumin molecules (proteins) that can be used to form cell matrices (e.g., stem cell matrices) of the invention. In many instances, this means that the Albumin employed will have functional charcteristics which allow for the formation of matrices having properties described herein. This includes proteins which have one or more functional domains of Albumin and proteins with sufficient sequence homology such that they contain one or more functional activities which allows for these proteins to participate in the function of matrices of the invention.

Thus, the term “Albumin” encompasses naturally occurring isoforms, such as those that occur in different tissues in the body.

An Albumin for use in the invention may be purified from any selected species, including without limitation human (Homo sapiens) and non-human primates. Non-human primates include without limitation Allenopithecus nigroviridis (Allen's swamp monkey), Allocebus trichotis (hairy-eared mouse lemur), Alouatta sp. (howler monkey), Alouatta seniculus (red howler), Aotus sp. (owl monkey), Arctocebus sp. (angwantibo), Ateles sp. (spider monkey), Ateles paniscus (black spider monkey), Avahi sp. (woolly indri), Brachyteles sp. (muriqui), Cacajao sp. (uakari), Callicebus sp. (titi), Callicebus moloch (dusky titi), Callimico goeldii (Goeldi's marmoset), Callithrix sp. (marmoset), Callithrix jacchus (common marmoset), Callithrix pygmaea (pygmy marmoset), Cebus sp. (capuchin monkey), Cebus apella (tufted capuchin), Cercocebus sp. (mangabey), Cercocebus atys (sooty mangabey), Cercopithecus sp. (guenon), Chemogaleus sp. (dwarf lemur), Chiropotes sp. (bearded saki), Chlorocebus sp. (vervet), Colobus sp. (black-and-white colobus), Daubentonia madagascariensis (aye-aye), Erythrocebus patas (patas monkey), Eulemur sp. (brown lemur), Euoticus sp. (needle-clawed bushbaby), Galago sp. (lesser bushbaby), Gorilla sp. (gorilla). Hapalemur sp. (bamboo lemur), Hoolock sp. (hoolock gibbon), Hylobates sp. (gibbon), Indri indri (indri), Lagothrix sp. (woolly monkey), Lemur catta (ring-tailed lemur), Leontopithecus sp. (lion tamarin), Leontopithecus chrysomelas (golden-headed lion tamarin), Leontopithecus rosalia (golden lion tamarin), Lepilemur sp. (sportive lemur), Lophocebus sp. (crested mangabey), Loris sp. (slender loris), Macaca sp. (macaque), Macaca arctoides (stump-tailed macaque), Macaca fascicularis (long-tailed macaque), Macaca fuscata (Japanese macaque), Macaca mulatta (rhesus macaque), Macaca nemestrina (pigtail macaque), Macaca nigra (crested black macaque), Mandrillus sp. (drill), Microcebus sp. (mouse lemur), Miopithecus sp. (talapoin), Mirza sp. (giant mouse lemur), Nasalis larvatus (proboscis monkey), Nomascus sp. (crested gibbon), Nomascus leucogenys (white-cheeked gibbon), Nycticebus sp. (slow loris), Oreonax flavicauda (yellow-tailed woolly monkey), Otolemur sp. (greater galago), Pan paniscus (bonobo), Pan troglodytes (chimpanzee), Papio sp. (baboon), Papio anubis (olive baboon), Papio cynocephalus (yellow baboon), Perodicticus potto (potto), Phaner sp. (fork-marked lemur), Piliocolobus sp. (red colobus), Pithecia sp. (saki monkey), Pongo sp. (orangutan), Presbytis sp. (surili), Procolobus verus (olive colobus), Prolemur simus (greater bamboo lemur), Propithecus sp. (sifaka), Propithecus diadema (diademed sifaka), Pygathrix sp. (douc), Rhinopithecus sp. (snub-nosed monkey), Rhinopithecus roxellana (golden snub-nosed monkey), Saguinus sp. (tamarin), Saguinus oedipus (cotton-top tamarin), Saimiri sp. (squirrel monkey), Semnopithecus sp. (gray langur), Simias concolor (pig-tailed langur), Symphalangus syndactylus (siamang), Tarsius sp. (tarsier), Theropithecus gelada (gelada baboon), Trachypithecus sp. (purple-faced langur) and Varecia sp. (ruffed lemur).

The term “Albumin” also encompasses synthetic molecules, such as synthetic peptides derived from full-length albumin proteins, albumin molecules produced by recombinant DNA technology, and the like. Synthetic peptides, comprising from 10 to about 200 amino acids can be synthesized in vitro according to methods known in the art. After amino acids are chemically polymerized in a sequence derived from the full peptide sequence of Albumin, synthetic peptides are purified and/or chemically modified (e.g., glycosylation, cross-linking).

Albumin can be produced using recombinant DNA technology. A gene for an Albumin protein is cloned directly or stepwise into an expression vector. The Albumin gene is induced (stimulated to express), resulting in increased synthesis of the Albumin protein. The Albumin may be part of a fusion protein.

Thus, the term “Albumin” encompasses naturally occurring isoforms of albumin.

The term “Albumin” also encompasses synthetic molecules, such as synthetic peptides derived from full-length albumin proteins, albumin molecules produced by recombinant DNA technology, and the like.

Non-limiting examples of Albumin preparations that can be used to practice the invention include:

1. Albuminar®-25 (Human Albumin, 25%; ZLB Behring LLC, Kankakee, Ill.), a sterile aqueous solution of albumin obtained from large pools of adult human venous plasma by low temperature controlled fractionation according to the Cohn process. It is stabilized with 0.02 M sodium acetyltryptophanate and 0.02 M sodium caprylate and pasteurized at 60° C. for 10 hours.

2. AlbuRx™ 25 (Human Albumin, 25%; ZLB Behring AG, Berne, Switzerland) is a sterile aqueous solution of human albumin suitable for intravenous administration containing the albumin component of human blood. This product is prepared from the plasma of U.S. donors. The product has been produced by alcohol fractionation and has been heated for 10 hours at 60° C. for inactivation of infectious agents. The results of virus validation studies have shown that the manufacturing process, particularly alcohol fractionation, eliminates enveloped and non-enveloped viruses. The aluminum content does not exceed 200 μg/L. The solution is stabilized with 0.08 millimole of sodium acetyltryptophanate plus 0.08 millimole of sodium caprylate per gram of albumin. The solution contains no preservative.

3. Albutein® 25% (Grifols) is a sterile aqueous solution for single dose intravenous administration containing 25% human albumin (weight/volume). Albutein® 25% is prepared by a cold alcohol fractionation method from pooled human plasma obtained from venous blood. The product is stabilized with 0.08 millimole sodium caprylate and 0.08 millimole sodium acetyltryptophanate per gram of albumin. Albutein® 25% solution is osmotically equivalent to five times its volume of normal human plasma. Albutein® 25% solution contains 130-160 milliequivalents of sodium ion per liter and has a pH of 6.9±0.5. The product contains no preservatives.

Fibronectin-Like Molecules

As used herein, the term “Fibronectin-like molecules” is meant to encompass any molecules (proteins) that can be used to form cell matrices (e.g., stem cell matrices) of the invention, such as, for example, vitronectin. In many instances, this means that the fibronectin-like molecules employed will have functional charcteristics which allow for the formation of matrices having properties described herein. This includes proteins which have one or more functional domains of vitronectin and proteins with sufficient sequence homology such that they contain one or more functional activity which allows for these proteins to participate in the function of matrices of the invention. In particular embodiments of the invention, where Fibronectin is used, Fibronectin-like molecules may be used in addition or in the place of Fibronectin.

Formulations

In many instances, either or both of the Fibronectin and Albumin components of the cell matrices, the cell cultures (e.g., stem cell cultures), matrix-forming and other formulations of the invention are at least about 95% pure, more preferably greater than 99% pure, and most preferably greater than 99.9% pure. A substance in a composition of matter is 95% pure when that substance represents 95% of the mass of the composition, excluding water.

Preferably, either or both of the Fibronectin and Albumin components of the cell matrices which may be cell matrices such as primary cell matrices or stem cell matrices, the cell cultures which may be cell cultures such as primary cell cultures or stem cell cultures, matrix-forming and other formulations of the invention are at least about 80% homogenous, more preferably greater than 90% homogenous, and most preferably greater than 99% homogenous. (The members of a population of any given molecular species are not necessarily identical; by way of non-limiting example, mild proteolysis of a protein produces a series of isoforms that are one or more amino acids shorter than the undigested, full-length protein. A substance is 80% homogenous when 80% of the molecules of that substance are identical to each other.)

Most preferably, both of the Fibronectin and Albumin components are greater than 99.9% pure and greater than 99% homogenous. Production of proteins of this quality often requires recombinant DNA technology (i.e., cloning and exogenous expression of a gene encoding a fibronectin or albumin polypeptide).

In some embodiments of this and other aspects of the invention, the Fibronectin is plasma fibronectin, including without limitation human plasma fibronectin (HPFN).

In some embodiments of this and other aspects of the invention, the Albumin is serum albumin, including without limitation human serum albumin (HSA).

Thus, in any aspect or embodiment of the invention, the cell matrix, which may be a cell matrix such as a primary cell matrix and/or stem cell matrix, could, by way of non-limiting example, comprise, consist of, or consist essentially of HPFN as the Fibronectin component and HSA as the Albumin component. Such a combination is particularly suited for the culture of cells which may be cells such as human primary cells or human stem cells. For other, non-human species, including without limitation primates, the invention might be practiced by using Fibronectin and Albumin derived from the same species as the cell grown on the matrix, which may be cells such as primary cells or stem cells. However, for any given species, it may not be necessary to make such adjustments; human Fibronectin and/or human Albumin may work well, or at least well enough, with cells, which may be cells such as primary cells or stem cells, derived from non-human primates and other animals.

In some embodiments of this and other aspects of the invention, the stem cell matrices of the invention comprise, consist of, or consist essentially of:

(a) HPFN, at a concentration of from about 0.005 mg/ml to about 5 mg/ml (e.g., from about 0.005 mg/ml to about 2 mg/ml, from about 0.005 mg/ml to about 1 mg/ml, from about 0.005 mg/ml to about 0.5 mg/ml, from about 0.005 mg/ml to about 0.1 mg/ml, from about 0.005 mg/ml to about 0.05 mg/ml, from about 0.01 mg/ml to about 5 mg/ml, from about 0.05 mg/ml to about 5 mg/ml, from about 0.1 mg/ml to about 5 mg/ml, from about 0.1 mg/ml to about 2 mg/ml, from about 0.5 mg/ml to about 2 mg/ml, from about 0.5 mg/ml to about 5 mg/ml, from about 0.75 mg/ml to about 2 mg/ml, from about 0.75 mg/ml to about 5 mg/ml, from about 1.0 mg/ml to about 2 mg/ml, etc.).

(b) HSA, at a concentration of from about 0.01% to about 5% (e.g., from about 0.01% to about 3%, from about 0.01% to about 2%, from about 0.01% to about 3%, from about 0.01% to about 0.75%, from about 0.01% to about 0.5%, from about 0.01% to about 0.25%, from about 0.1% to about 5%, from about 0.5% to about 5%, from about 0.5% to about 3%, from about 0.75% to about 3%, from about 0.75% to about 5%, from about 1.0% to about 2%, from about 1.0% to about 3%, from about 1.0% to about 5%, etc.).

In some embodiments, cell matrices, which may be cell matrices such as primary cell matrices or stem cell matrices, such as ESC matrices, of the invention include formulations that comprise, consist of, or consist essentially of a composition with components at concentrations selected from the group consisting of:

(a) 0.01 mg/ml HPFN and 0.02% HSA,

(b) 0.04 mg/ml HPFN and 0.08% HSA,

(c) 0.09 mg/ml HPFN and 0.2% HSA,

(d) 0.4 mg/ml HPFN and 0.8% HSA, and

(e) 0.92 mg/ml HPFN and 2% HSA.

In some embodiments, the cell matrices, which may be cell matrices such as primary cell matrices or stem cell matrices, such as ESC matrices, and other compositions of matter of the invention are serum-free. A formulation is “serum-free” when it has been assembled without the addition of sera of any type from any source.

In some embodiments, the cell matrices, which may be cell matrices such as primary cell matrices or stem cell matrices, such as ESC matrices, and other compositions of matter of the invention are xeno-free. A composition of matter is said to be “xeno-free” when it is devoid of substances from any animal other than the species of animal from which the cells are derived.

In some embodiments, the cell matrices, which may be cell matrices such as primary cell matrices or stem cell matrices, such as ESC matrices, and other compositions of matter of the invention are completely defined. As the term is used herein, a formulation is an assembled (man-made) composition of matter. By definition, a formulation is “defined” in the sense that the identity and concentration of its components are known. However, in cell culture media and systems, incompletely defined formulations are often used. For example, some cell culture formulations are or comprise ingredients that are incompletely characterized extracts from biological sources; non-limiting examples of such extracts include sera, Matrigel™ and other stem cell matrices derived from biological sources, and conditioned media. An “incompletely defined” formulation is one that comprises one or more ingredients that is (1) an uncharacterized substance, such as a biological extract; (2) less than about 90% pure; (3) less than about 80% homogenous; and (4) present in a unknown amount and/or concentration (unquantified). By way of comparison, a formulation is “completely defined” when each of the ingredients used to prepare it is (1) a known (characterized) substance; (2) at least about 90% pure; (3) at least about 80% homogenous; and (4) quantified (present in a known amount and/or concentration). Often, in order to obtain components of the requisite purity and homogeneity, it is often useful or necessary to use synthetic ingredients, such as synthetic chemical compounds, recombinant nucleic acids and proteins, and the like.

In some embodiments, the cell matrices (which may be cell matrices such as primary cell matrices or stem cell matrices, such as ESC matrices), matrix-forming formulations, and other compositions of matter of the invention are sterilized to prevent unwanted contamination. Sterilization may be accomplished, for example, by ultraviolet light, filtration, or heat. Antibiotics may also be added, particularly during incubation, to prevent the growth of bacteria, fungi and other undesired micro-organisms. Such antibiotics include, by way of non-limiting example, gentamicin, streptomycin, penicillin, amphotericin and ciprofloxacin.

In some embodiments, the cell matrices (which may be cell matrices such as primary cell matrices or stem cell matrices, such as ESC matrices), matrix-forming formulations, and other compositions of matter of the invention further comprise one or more buffering agents. Buffering agents that may be included in the culture media of the present invention include, but are not limited to, buffered saline solutions such as phosphate-buffered saline (PBS) formulations, Tris-buffered saline (TBS) formulations, HEPES-buffered saline (HBS) formulations, Hanks Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, Kao and Michayluk's Basal Salt Solutions, and the like. Formulations for these buffers, which are commercially available, as well as for many other commonly used buffers, are well-known in the art and may be found for example in the GIBCO/BRL Catalogue and Reference Guide (Gibco/Invitrogen, Carlsbad, Calif., US), in the DIFCO Manual (DIFCO; Norwood, Mass., US), and in the Sigma Cell Culture Catalogues for animal and plant cell culture (Sigma; St. Louis, Mo., US)

Cell Cultures

In another embodiment, the invention provides, in part, cell cultures, including without limitation, cell cultures which may be cell cultures such as primary cell cultures or stem cell cultures, such as ESC, comprising cell matrices (which may be cell matrices such as primary cell matrices or stem cell matrices, such as ESC matrices) that comprise or consist essentially of Fibronectin and Albumin.

A stem cell culture of the invention comprises:

(a) stem cells;

(b) culture media; and

(c) a stem cell matrix, wherein said stem cell matrix:

(1) consists essentially of Fibronectin and Albumin;

(2) comprises amounts of Fibronectin and Albumin sufficient for the formation of a cellular matrix; and

(3) supports the growth of stem cells in the presence of culture media.

A primary cell culture of the invention comprises:

(a) primary cells;

(b) culture media; and

(c) a primary cell matrix, wherein said primary cell matrix:

(1) consists essentially of Fibronectin and Albumin;

(2) comprises amounts of Fibronectin and Albumin sufficient for the formation of a cellular matrix; and

(3) supports the growth of primary cells in the presence of culture media.

In some embodiments of the invention, the primary cells of the primary cell cultures may be keratinocytes, which include without limitation, human dermal keratinocytes. In other embodiments of the invention, the primary cells may be fibroblast cells, which include without limitation, human fetal fibroblast cells. In some embodiments of the invention, the stem cells of the stem cell cultures of the invention are embryonic stem cells. The source of embryonic stem cells can include without limitation mammals, including non-human primates and humans. Non-limiting examples of human embryonic stem cells include lines BG01, BG02, BG03, BG01v, CHA-hES-1, CHA-hES-2, FCNCBS1, FCNCBS2, FCNCBS3, H1, H7, H9, H13, H14, HSF-1, H9.1, H9.2, HES-1, HES-2, HES-3, HES-4, HES-5, HES-6, hES-1-2, hES-3-0, hES-4-0, hES-5-1, hES-8-1, hES-8-2, hES-9-1, hES-9-2, hES-101, hICM8, hICM9, hICM40, hICM41, hICM42, hICM43, HSF-6, HUES-1, HUES-2, HUES-3, HUES-4 HUES-5, HUES-6, HUES-7 HUES-8, HUES-9, HUES-10, HUES-11, HUES-12, HUES-13, HUES-14, HUESS-15, HUES-16, HUES-17, 13, 14, 16, 13.2, 13.3, 16.2, J3, J3.2, MB01, MB02, MB03, Miz-hES1, RCM-1, RLS ES 05, RLS ES 07, RLS ES 10, RLS ES 13, RLS ES 15, RLS ES 20, RLS ES 21, SA01, SA02, and SA03. In some embodiments of the invention, the stem cells of the stem cell cultures of the invention are induced pluripotent stem cells.

In some embodiments, the cell cultures which may be cell cultures such as primary cell cultures or stem cell cultures of the invention are serum-free. In some these embodiments, a serum-free primary cell matrix of the invention is used in conjunction with a primary cell serum-free media (SFM). Suitable SFM include without limitation (a) EpiLife® Serum Free Culture Medium supplemented with EpiLife® Defined Growth Supplement and (b) Defined Keratinocyte-SFM supplemented with Defined Keratinocyte-SFM Growth Supplement, all commercially available from Gibco/Invitrogen (Carlsbad, Calif., US). In some of these embodiments, a serum-free stem cell matrix of the invention is used in conjunction with stem cell SFM. Suitable SFM include without limitation StemPro® hESC Serum Free Media (SFM) supplemented with basic fibroblast growth factor and β-mercaptoethanol, Knockout™ D-MEM supplemented with Knockout™ Serum Replacement (SR), StemPro® MSC SFM and StemPro® NSC SFM, all commercially available from Gibco/Invitrogen (Carlsbad, Calif., US).

In some embodiments, the cell cultures which may be cell cultures such as primary cell cultures or stem cell cultures of the invention are xeno-free. In these embodiments, a xeno-free cell matrix which may be a cell matrix such as a primary cell matrix or stem cell matrix of the invention is used in conjunction with xeno-free media.

In some embodiments, the cell cultures which may be cell culture such as primary cell cultures or stem cell cultures of the invention comprise a culture medium that is a conditioned culture medium. In other embodiments, the culture medium is an unconditioned culture medium.

In some embodiments of the invention, the cell cultures which may be cell cultures such as primary cell cultures or stem cell cultures of the invention are completely defined. In these embodiments, a completely defined cell matrix which may be a cell matrix such as a primary cell matrix or stem cell matrix of the invention is used conjunction with completely defined media. Suitable media include without limitation, for primary cells, EpiLife® Serum Free Culture Medium supplemented with EpiLife® Defined Growth Supplement, and, for stem cells, StemPro® hESC SFM, all commercially available from Gibco/Invitrogen, Carlsbad, Calif., US.

In preferred embodiments of aspects of the invention that comprise one or more stem cell cultures, the stem cells have one or more desirable characteristics associated with robust stem cell culture. Non-limiting examples of such desirable characteristics include:

(a) the ability to proliferate in culture for at least about 50 passages while maintaining the potential to differentiate into derivatives of endoderm, mesoderm, and ectoderm tissues;

(b) the ability to proliferate in culture for at least about 50 passages without developing an altered karyotype;

(c) expression of the expression transcription factor Oct4; and

(d) the presence of one or more surface antigens specific to stem cells.

Non-limiting examples of surface antigens specific to stem cells are selected from the list consisting of: (a) SSEA-3; (b) SSEA-4; (c) TRA-1-60; and (d) TRA-1-81. Typically, these proteins are detected using antibodies specific for each (Hoffman and Carpenter, Nat. Biotechnol. 23:699-708, 2005).

Matrix-Forming Formulations

In another embodiment, the invention provides, in part, formulations for forming the cell matrices which may be cell matrices such as primary cell matrices and stem cell matrices of the invention (matrix-forming formulations).

A matrix-forming formulation of the invention consists essentially of Fibronectin and Albumin, wherein said Fibronectin and said Albumin are present at concentrations effective for forming a cellular matrix.

In some embodiments, the Fibronectin is plasma fibronectin, including without limitation human plasma fibronectin (HPFN).

In some embodiments, the Albumin is serum albumin, including without limitation human serum albumin (HSA).

In some embodiments, a matrix-forming formulation of the invention consists essentially of:

(a) HPFN, at a concentration of from about 0.005 mg/ml to about 5 mg/mL, and

(b) HSA, at a concentration of from about 0.01% to about 5%.

In some embodiments, the matrix-forming formulations of the invention are formulations that comprise or consist essentially of a composition selected from the group consisting of:

(a) 0.01 mg/ml HPFN and 0.02% HSA,

(b) 0.04 mg/ml HPFN and 0.08% HSA,

(c) 0.09 mg/ml HPFN and 0.2% HSA.

(d) 0.4 mg/ml HPFN and 0.8% HSA, and

(e) 0.92 mg/ml HPFN and 2% HSA.

In some embodiments, the matrix-forming formulations of the invention are serum-free.

In some embodiments, the matrix-forming formulations of the invention are xeno-free.

In some embodiments, the matrix-forming formulations of the invention are completely defined.

Articles of Manufacture

In another aspect, the invention provides, in part, articles of manufacture.

Embodiments of this aspect of the invention include non-mechanical articles, as well as mechanical articles (devices), and kits.

Articles of manufacture of the invention include without limitation articles comprising one or more cell matrices which may be cell matrices such as primary cell matrices or stem cell matrices, one or more cell cultures which may be cell cultures such as primary cell cultures or stem cell cultures of the invention, and/or one or more matrix-forming or other formulations of the invention.

In one embodiment of this and other aspects of the invention, the non-mechanical article of manufacture of the invention is a culture vessel having a substrate surface that is coated with a cell matrix which may be a cell matrix such as a primary cell matrix or stem cell matrix of the invention. Culture vessels include without limitation flasks, Petri dishes, and multiwell plates.

In another embodiment, the non-mechanical article of manufacture of the invention is a biological implant that comprises a stem cell culture, wherein the culture comprises a stem cell matrix of the invention, and wherein the implant has no mechanical parts and instead functions through the use of a passive mechanism. One type of passive mechanism is diffusion, which is the driving force behind the non-mechanical article of manufacture commonly known as the “nicotine patch”.

In another embodiment, the mechanical article of manufacture (device) of the invention is a biological implant that comprises one or more mechanical parts (e.g., a micropump) and a stem cell culture, wherein the culture comprises a stem cell matrix of the invention.

In another embodiment, the article of manufacture of the invention is a device comprising one or more mechanical parts and a stem cell matrix of the invention.

In another embodiment, the article of manufacture of the invention is a kit (kits of the invention are discussed in more detail elsewhere herein).

In these and other aspects and embodiments of the invention, the invention provides, in part, an article of manufacture comprising a cell matrix which may be a cell matrix such as a primary cell matrix or stem cell matrix, wherein said cell matrix consists essentially of Fibronectin and Albumin.

In some embodiments, the cell matrices which may be cell matrices such as primary cell matrices or stem cell matrices, or cell cultures which may be cell cultures such as primary cell cultures or stem cell cultures present in the articles of manufacture are serum-free. Optionally, the entire article of manufacture is serum-free.

In some embodiments, the cell matrices which may be cell matrices such as primary cell matrices or stem cell matrices, or cell cultures which may be cell cultures such as primary cell cultures or stem cell cultures present in the articles of manufacture are xeno-free. Optionally, the entire article of manufacture is xeno-free.

In various embodiments, the Fibronectin is plasma fibronectin; for human stem cells, the Fibronectin is preferably a human fibronectin, most preferably human plasma fibronectin (HPFN).

In various embodiments, the Albumin is serum albumin; for human stem cells, the Albumin is preferably a human albumin, most preferably human serum albumin (HSA).

In other embodiments, the invention provides, in part, an article of manufacture comprising a culture of primary cells, said culture comprising:

(a) a population of primary cells;

(b) a culture medium; and

(c) a primary cell matrix, wherein said primary cell matrix:

(1) consists essentially of Fibronectin and Albumin;

(2) comprises amounts of Fibronectin and Albumin sufficient for the formation of a cellular matrix; and

(3) supports the growth of primary cells in the presence of culture media.

In other embodiments, the invention provides, in part, an article of manufacture comprising a culture of stem cells, said culture comprising:

(a) a population of stem cells;

(b) a culture medium; and

(c) a stem cell matrix, wherein said stem cell matrix:

(1) consists essentially of Fibronectin and Albumin;

(2) comprises amounts of Fibronectin and Albumin sufficient for the formation of a cellular matrix; and

(3) supports the growth of stem cells in the presence of culture media.

In some embodiments, the primary cells in the articles of manufacture comprising the primary cells are keratinocytes, which may include without limitation, human dermal keratinocytes. In some embodiments of the invention, the primary cells in the articles of manufacture comprising the primary cells are fibroblast cells, which may include without limitation, human fetal fibroblast cells. The source of primary cells can include without limitation mammals, including non-human primates and humans. In some embodiments, the stem cells in articles of manufacture comprising stem cell cultures of the invention are embryonic stem cells. The source of embryonic stem cells can include without limitation mammals, including non-human primates and humans. Non-limiting examples of human embryonic stem cells include lines BG01, BG02, BG03, BG01v, CHA-hES-1, CHA-hES-2, FCNCBS1, FCNCBS2, FCNCBS3, H1, H7, H9, H13, H14, HSF-1, H9.1, H9.2, HES-1, HES-2, HES-3, HES-4, HES-5, HES-6, hES-1-2, hES-3-0, hES-4-0, hES-5-1, hES-8-1, hES-8-2, hES-9-1, hES-9-2, hES-101, hICM8, hICM9, hICM40, hICM41, hICM42, hICM43, HSF-6, HUES-1, HUES-2, HUES-3, HUES-4 HUES-5, HUES-6, HUES-7 HUES-8, HUES-9, HUES-10, HUES-11, HUES-12, HUES-13, HUES-14, HUESS-15, HUES-16, HUES-17, 13, 14, 16, 13.2, 13.3, 16.2, J3, J3.2, MB01, MB02, MB03, Miz-hES1, RCM-1, RLS ES 05, RLS ES 07, RLS ES 10, RLS ES 13, RLS ES 15, RLS ES 20, RLS ES 21, SA01, SA02, and SA03. In some embodiments of the invention, the stem cells in the articles of manufacture comprising stem cells are induced pluripotent stem cells. The source of induced pluripotent stem cells can include without limitation cells from mammals, including non-human primates and humans.

In some embodiments, the stem cell cultures in articles of manufacture comprising cell cultures which may be cell cultures such as primary cell cultures or stem cell cultures of the invention are serum-free. In these embodiments, a cell matrix which may be a cell matrix such as a serum-free primary cell matrix or serum-free stem cell matrix of the invention is used in conjunction with serum-free media (SFM). Suitable SFM for primary cells include without limitation (a) EpiLife® Serum Free Culture Medium supplemented with EpiLife® Defined Growth Supplement and (b) Defined Keratinocyte-SFM supplemented with Defined Keratinocyte-SFM Growth Supplement, all commercially available from Gibco/Invitrogen (Carlsbad, Calif., US). Suitable SFM for stem cells include without limitation (a) StemPro® hESC Serum Free Media (SFM) supplemented with basic fibroblast growth factor and β-mercaptoethanol, and (b) Knockout™ D-MEM supplemented with Knockout™ Serum Replacement (SR), StemPro® MSC SFM and StemPro® NSC SFM, all commercially available from Gibco/Invitrogen (Carlsbad, Calif., US). In further embodiments, the entire article of manufacture comprising a cell culture which may be a cell culture such as a primary cell culture or stem cell culture of the invention is serum-free.

In some embodiments, the primary cell cultures in articles of manufacture comprising primary cell cultures of the invention are xeno-free. In these embodiments, a xeno-free primary cell matrix of the invention is used in conjunction with xeno-free media. In further embodiments, the entire article of manufacture comprising a primary cell culture of the invention is xeno-free. In some embodiments, the stem cell cultures in articles of manufacture comprising stem cell cultures of the invention are xeno-free. In these embodiments, a xeno-free stem cell matrix of the invention is used in conjunction with xeno-free media. In further embodiments, the entire article of manufacture comprising a stem cell culture of the invention is xeno-free.

In some embodiments, the culture media in articles of manufacture comprising primary cell cultures or stem cell cultures of the invention are conditioned culture media; in other embodiments, the culture media are unconditioned culture media.

In some embodiments, the culture media in articles of manufacture comprising primary cell cultures of the invention are completely defined. In these embodiments, a completely defined primary cell matrix of the invention is used conjunction with completely defined media. Suitable media for primary cells include without limitation (a) EpiLife® Serum Free Culture Medium supplemented with EpiLife® Defined Growth Supplement and (b) Defined Keratinocyte-SFM supplemented with Defined Keratinocyte-SFM Growth Supplement, all commercially available from Gibco/Invitrogen (Carlsbad, Calif., US). In some embodiments, the culture media in articles of manufacture comprising stem cell cultures of the invention are completely defined. In these embodiments, a completely defined stem cell matrix of the invention is used conjunction with completely defined media. Suitable media for stem cells include without limitation StemPro® hESC SFM (Gibco/Invitrogen, Carlsbad, Calif., US).

In preferred embodiments of aspects of the invention that comprise one or more stem cell cultures, the stem cells have one or more desirable characteristics associated with robust stem cell culture. Non-limiting examples of such desirable characteristics include:

(a) the ability to proliferate in culture for at least about 50 passages while maintaining the potential to differentiate into derivatives of endoderm, mesoderm, and ectoderm tissues;

(b) the ability to proliferate in culture for at least about 50 passages without developing an altered karyotype;

(c) expression of the expression transcription factor Oct4; and

(d) the presence of one or more surface antigens specific to stem cells. Non-limiting examples of surface antigens specific to stem cells include (a) SSEA-3, (b) SSEA-4, (c) TRA-1-60, and (d) TRA-1-81.

Kits

In another embodiment, the invention provides, in part, kits.

Generally, such kits are used in the course of culturing cells which may be cells such as primary cells or stem cells or preparing materials necessary therefor.

In some embodiments, the invention provides, in part, a kit comprising a carrier means compartmentalized to receive in close confinement therein one or more container means, wherein a first container means contains a matrix-forming formulation according to the present invention.

In some embodiments, the invention provides, in part, a kit comprising a carrier means compartmentalized to receive in close confinement therein one or more container means, wherein a first container means contains a stem cell matrix according to the present invention.

In some embodiments, the invention provides, in part, a kit comprising a carrier means compartmentalized to receive in close confinement therein one or more container means, wherein a first container means contains a cell culture which may be a cell culture such as a primary cell culture or stem cell culture according to the present invention.

Non-limiting examples of carrier means include boxes, cartons, bags, and the like.

Non-limiting examples of container means include bottles, bags, pouches, vials, tubes, ampules, jars, and the like.

Optionally, kits of the invention comprise additional container means that contain components that include, by way of non-limiting example, culture media, buffers, salts, energy sources, antibodies and the like.

A kit typically also comprises a set of written instructions for the preparation, storage and/or use of its contents.

Methods of Making

In another aspect, the invention provides, in part, methods of making the compositions of matter and articles of manufacture of the invention.

In an exemplary embodiment of this aspect of the invention, the invention provides, in part, a method of preparing an article of manufacture comprising a surface coated with a cell matrix which may be a cell matrix such as a primary cell matrix or stem cell matrix, wherein said cell matrix which may be a cell matrix such as a primary cell matrix or stem cell matrix consists essentially of Fibronectin and Albumin at concentrations effective for forming a cellular matrix, said method comprising the following steps, carried out simultaneously or in any order:

(a) coating a substrate surface of said article of manufacture with Fibronectin; and

(b) coating an overlapping portion of said substrate surface of said article of manufacture with Albumin.

A “substrate surface” is a position on or a portion of a surface on an article of manufacture that is, or is intended to be, coated with a cell matrix which may be a cell matrix such as a primary cell matrix or stem cell matrix. In a culture vessel, the substrate surface is typically the lower interior surface(s) of the vessel. Non-limiting examples of culture vessels include a flask, a Petri dish, and a multiwell plate.

In some embodiments of this aspect of the invention, the steps of (a) coating a substrate surface of said article of manufacture with Fibronectin, and (b) coating an overlapping portion of said substrate surface of said article of manufacture with Albumin, are carried out simultaneously.

In another embodiment of this aspect of the invention, the invention provides, in part, a method of preparing an article of manufacture comprising a surface coated with a cell matrix which may be a cell matrix such as a primary cell matrix or stem cell matrix, wherein said method comprises the step of contacting said substrate surface with a matrix-forming that consists essentially of Fibronectin and Albumin, wherein said Fibronectin and said Albumin are present at concentrations effective for forming a cellular matrix.

In some embodiments of the methods of the invention, the Fibronectin is plasma fibronectin, including without limitation human plasma fibronectin (HPFN). In some embodiments, the Albumin is serum albumin, including without limitation human serum albumin (HSA).

In some embodiments of the methods of the invention, a matrix-forming formulation of the invention consists essentially of:

(a) HPFN, at a concentration of from about 0.005 mg/ml to about 5 mg/mL, and

(b) HSA, at a concentration of from about 0.01% to about 5%.

In some embodiments of the methods of the invention, the matrix-forming formulations of the invention are formulations that comprise or consist essentially of a composition selected from the group consisting of:

(a) 0.01 mg/ml HPFN and 0.02% HSA,

(b) 0.04 mg/ml HPFN and 0.08% HSA,

(c) 0.09 mg/ml HPFN and 0.2% HSA.

(d) 0.4 mg/ml HPFN and 0.8% HSA, and

(e) 0.92 mg/ml HPFN and 2% HSA.

In some embodiments of the methods of the invention, the matrix-forming formulations of the invention are serum-free.

In some embodiments of the methods of the invention, the matrix-forming formulations of the invention are xeno-free.

In some embodiments of the methods of the invention, the matrix-forming formulations of the invention are completely defined.

Methods of Use

In another aspect, the invention provides, in part, methods of using the compositions of matter and articles of manufacture of the invention.

In exemplary embodiments of this aspect of the invention, the invention provides, in part, methods of using compositions of matter and/or articles of manufacture of the invention to culture cells which may be cells such as primary cells or stem cells. Although they are described herein as a method of use, these methods also serve as methods of making other aspects of the invention, i.e., compositions of matter and articles of manufacture comprising cell cultures which may be cell cultures such as primary cell cultures or stem cell cultures.

In an exemplary embodiment of this aspect of the invention, the invention provides, in part, a method of generating a primary cell culture, comprising the following steps:

(a) generating a primary cell matrix in a culture vessel by contacting a substrate surface of said culture vessel with a matrix-forming formulation, wherein said matrix-forming formulation consists essentially of Fibronectin and Albumin at concentrations effective for forming a cellular matrix, and removing excess formulation, thereby generating a primary cell matrix on said substrate surface;

(b) adding a culture medium and primary cells to said culture vessel in amounts sufficient to allow said stem cells and medium to contact said primary cell matrix; and

(c) incubating said culture vessel.

In some embodiments, the matrix-forming formulation, primary cell matrix, culture media and culture vessel are serum-free; when all of the preceding are serum-free, the primary cell culture itself is also serum-free. Suitable serum-free media (SFM) for primary cells include without limitation (a) EpiLife® Serum Free Culture Medium supplemented with EpiLife® Defined Growth Supplement and (b) Defined Keratinocyte-SFM supplemented with Defined Keratinocyte-SFM Growth Supplement, all commercially available from Gibco/Invitrogen (Carlsbad, Calif., US).

In some embodiments, the matrix-forming formulation, primary cell matrix, culture media and culture vessel are xeno-free; when all of the preceding are xeno-free, the primary cell culture itself is also xeno-free.

In some embodiments, the culture media comprising primary cell cultures of the invention are conditioned culture media; in other embodiments, the culture media are unconditioned culture media.

In some embodiments, the culture media comprising primary cell cultures of the invention are completely defined. In these embodiments, a completely defined stem cell matrix of the invention is used conjunction with completely defined media. Suitable media for primary cells include without limitation EpiLife® Serum Free Culture Medium supplemented with EpiLife® Defined Growth Supplement, commercially available from Gibco/Invitrogen (Carlsbad, Calif., US).

In some embodiments, the primary cells in the primary cell cultures are keratinocytes, which include without limitation, human dermal keratinocytes. In some embodiments, the primary cells are fibroblast cells, which include without limitation, human fetal fibroblast cells. The source of primary cells can include without limitation mammals, including non-human primates and humans.

In another exemplary embodiment of this aspect of the invention, the invention provides, in part, a method of generating a stem cell culture, comprising the following steps:

(a) combining in a culture vessel, simultaneously or in any order:

(1) a stem cell matrix, wherein said stem cell matrix consists essentially of Fibronectin and Albumin at concentrations effective for forming a cellular matrix;

(2) a culture medium; and

(3) stem cells; and

(b) incubating said culture vessel.

In another exemplary embodiment of this aspect of the invention, the invention provides, in part, a method of generating a stem cell culture, comprising the following steps:

(a) generating a stem cell matrix in a culture vessel by contacting a substrate surface of said culture vessel with a matrix-forming formulation, wherein said matrix-forming formulation consists essentially of Fibronectin and Albumin at concentrations effective for forming a cellular matrix, and removing excess formulation, thereby generating a stem cell matrix on said substrate surface;

(b) adding a culture medium and stem cells to said culture vessel in amounts sufficient to allow said stem cells and medium to contact said stem cell matrix; and

(c) incubating said culture vessel.

In various embodiments, the matrix-forming formulation, stem cell matrix, culture media and culture vessel are serum-free; when all of the preceding are serum-free, the stem cell culture itself is also serum-free. Suitable serum-free media (SFM) include without limitation StemPro® hESC Serum Free Media (SFM) supplemented with basic fibroblast growth factor and β-mercaptoethanol, Knockout™ D-MEM supplemented with Knockout™ Serum Replacement (SR), StemPro® MSC SFM and StemPro® NSC SFM, all commercially available from Gibco/Invitrogen (Carlsbad, Calif., US).

In various embodiments, the matrix-forming formulation, stem cell matrix, culture media and culture vessel are xeno-free; when all of the preceding are xeno-free, the stem cell culture itself is also xeno-free.

In some embodiments, the culture media comprising stem cell cultures of the invention are conditioned culture media; in other embodiments, the culture media are unconditioned culture media.

In some embodiments, the culture media comprising stem cell cultures of the invention are completely defined. In these embodiments, a completely defined stem cell matrix of the invention is used conjunction with completely defined media. Suitable media include without limitation StemPro® hESC SFM (Gibco/Invitrogen, Carlsbad, Calif., US).

In some embodiments, the stem cells in the stem cell cultures are embryonic stem cells. The source of embryonic stem cells can include without limitation mammals, including non-human primates and humans. Non-limiting examples of human embryonic stem cells include lines BG01, BG02, BG03, BG01v, CHA-hES-1, CHA-hES-2, FCNCBS1, FCNCBS2, FCNCBS3, H1, H7, H9, H13, H14, HSF-1, H9.1, H9.2, HES-1, HES-2, HES-3, HES-4, HES-5, HES-6, hES-1-2, hES-3-0, hES-4-0, hES-5-1, hES-8-1, hES-8-2, hES-9-1, hES-9-2, hES-101, hICM8, hICM9, hICM40, hICM41, hICM42, hICM43, HSF-6, HUES-1, HUES-2, HUES-3, HUES-4 HUES-5, HUES-6, HUES-7 HUES-8, HUES-9, HUES-10, HUES-11, HUES-12, HUES-13, HUES-14, HUESS-15, HUES-16, HUES-17, 13, 14, 16, 13.2, 13.3, 16.2, J3, J3.2, MB01, MB02, MB03, Miz-hES1, RCM-1, RLS ES 05, RLS ES 07, RLS ES 10, RLS ES 13, RLS ES 15, RLS ES 20, RLS ES 21, SA01, SA02, and SA03. In some embodiments, the stem cells are induced pluripotent stem cells. The source of induced pluripotent stem cells can include without limitation cells from mammals, including non-human primates and humans.

In preferred embodiments, the stem cells have one or more desirable characteristics associated with robust stem cell culture. Non-limiting examples of such desirable characteristics include:

(a) the ability to proliferate in culture for at least about 50 passages while maintaining the potential to differentiate into derivatives of endoderm, mesoderm, and ectoderm tissues;

(b) the ability to proliferate in culture for at least about 50 passages without developing an altered karyotype;

(c) expression of the expression transcription factor Oct4; and

(d) the presence of one or more surface antigens specific to stem cells. Non-limiting examples of surface antigens specific to stem cells include (a) SSEA-3, (b) SSEA-4, (c) TRA-1-60, and (d) TRA-1-81.

In Vivo Uses

Bioresorbable sponges using the matrices of the invention can provide a temporary scaffolding for transplanted cells, and thereby allow the cells to secrete extracellular matrix of their own to enable, in the long term, a complete and natural tissue replacement. The macromolecular structure of these sponges is selected so that they are completely degradable and are eliminated, once they have achieved their function of providing the initial artificial support for the newly transplanted cells. For these sponges to be useful in cell transplantations, they must be highly porous with large surface/volume ratios to accommodate a large number of cells, they must be biocompatible, i.e., non-toxic to the cells that they carry and to the host tissue into which they are transplanted, they must be capable of promoting cell adhesion and allowing the retention of the differentiated function of attached cells.

The present invention thus also provides, in part, artificial organ equivalents which serve to provide the essential function of the organ which they are to replace fully or partially or whose function they are designed to augment. The artificial organ equivalents of the invention therefore comprise a bioresorbable sponge of the invention, as noted above, and representative cells of the organ, the cells having been grown on or within the sponge in vitro to the stage wherein they are active and at least about 10%, preferably about 50%, most preferably equivalent to the active cells of the organ and thereby the artificial organ is suitable for transplantation or implantation in all the various ways thereof as detailed above, into a patient in need thereof following organ damage, removal or dysfunction. Preferred embodiments of the artificial organ equivalents of the invention being artificial skin comprising a polysaccharide sponge of the invention and dermal fibroblast cells, as well as an artificial liver equivalent comprising a polysaccharide sponge of the invention and hepatocytes.

Instructions and Methods in Tangible Form

In another aspect, the invention provides instructions and methods in tangible form. Such instructions and methods may be for the preparation, storage and/or use of the invention in any of its various aspects and embodiments.

Non-limiting examples of suitable tangible forms include without limitation sheets of paper; the carrier means of a kit; a container, such as a box, bottles, vial, bag, or pouch; computer-readable digital files on computer-readable media such as compact discs (CDs), digital video discs (DVDs), thumb drives, micro-drives, computer-internal drives, computer-external drives, flash memory cards, and the like; portable data encoders/players such as laptop computers, PDAs, personal DVD players, i-Phones; digital audio players (DAPs) comprising computer-readable digital files; and the like.

A flash memory card is a solid-state electronic flash memory data storage device that has the desirable characteristics of having a high re-record-ability, power-free storage, small size, and durability. There are different types of flash memory cards, including without limitation: a PC card (PCMCIA, Personal Computer Memory Card International Association); a CompactFlash® (CF) card; a Memory Stick (MS); a Secure Digital (SD) card; a Miniature Card (MiniCard); a MultiMedia Card (MMC); a SmartMedia card; and an xD Picture Card.

A digital audio player (DAP) is a device that stores, organizes and plays digital audio (e.g., music) files. Although DAPs are often referred to as MP3 players, DAPs typically play many additional file formats. Some formats are proprietary, such as MP3, Windows Media Audio (WMA), and Advanced Audio Codec (AAC). An iPod is a specific type of a (proprietary) MP3 player.

Computer-readable digital files can be in any of a variety of formats, including without limitation those have any of the following extensions: .doc; .txt; .pdf; .jpg; .jpeg; .tiff; .giff; .psd; .wmv; .mpg; .mpeg; .swf; and .mp3.

Another suitable tangible form is the Internet, a global population of computers connected to each other. When a member of the internet “posts” or “publishes” (makes accessible to other users of the internet) a set of instructions of the invention, the scope of the tangible form known as the internet includes the publisher's computer, the publisher's server, and any computer connected to the internet that accesses (views or downloads) the published set of instructions.

The invention thus includes, as examples, instructions methods and/or instructions described herein or for using materials described herein. As an example, the invention includes kits which contain (A) (1) one or more (e.g., one, two, three, four, five, etc.) component described herein and/or (2) one or more components for use in methods described herein and (B) a printed sheet of paper which contains printed on it (1) methods described herein and/or (2) methods for using one or more component described herein.

Business Methods

The present invention also provides a system and method of providing company products to a party outside of the company, for example, a system and method for providing a customer or a product distributor a product of the company such as a kit containing a cell matrix. FIG. 1 provides a schematic diagram of a product management system. In practice, the blocks in FIG. 1 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The product management system as shown in FIG. 1 is exemplified by company 100, which receives input in the form of an order from a party outside of the company, e.g., distributor 150 or customer 140, to order department 126, or in the form of materials and parts 130 from a party outside of the company; and provides output in the form of a product delivered from shipping department 119 to distributor 150 or customer 140. Company 100 system is organized to optimize receipt of orders and delivery of a products to a party outside of the company in a cost efficient manner, particularly instructions or a kit of the present invention, and to obtain payment for such product from the party.

With respect to methods of the present invention, the term “materials and parts” refers to items that are used to make a device, other component, or product, which generally is a device, other component, or product that company sells to a party outside of the company. As such, materials and parts include, for example, proteins, membranes, lipids, salts, solid supports, buffers, etc. Devices are exemplified by cell matrices described herein. Other components are exemplified by instructions, including instructions for preparing and using cell matrices (e.g., instructions for cultivating cells on cell matrices). Other components also can be items that may be included in a kit. As such, it will be recognized that an item useful as materials and parts as defined herein further can be considered an “other component”, which can be sold by the company. The term “products” refers to devices, other components, or combinations thereof, including combinations with additional materials and parts, that are sold or desired to be sold or otherwise provided by a company to one or more parties outside of the company. Products are exemplified herein by cell matrices and kits (e.g., kits which can contain instructions according to the present invention).

Referring to FIG. 1, company 100 includes manufacturing 110 and administration 120. Devices 112 and other components 114 are produced in manufacturing 110, and can be stored separately therein such as in device storage 113 and other component storage 115, respectively, or can be further assembled and stored in product storage 117. Materials and parts 130 can be provided to company 100 from an outside source and/or materials and parts 114 can be prepared in company, and used to produce devices 112 and other components 116, which, in turn, can be assembled and sold as a product. Manufacturing 110 also includes shipping department 119, which, upon receiving input as to an order, can obtain products to be shipped from product storage 117 and forward the product to a party outside the company.

For purposes of the present invention, product storage 117 can store instructions, for example, for preparing and using cell matrices described herein, as well as combinations of such instructions and/or kits. Upon receiving input from order department 126, for example, that customer 140 has ordered such a kit and instructions, shipping department 119 can obtain from product storage 117 such kit for shipping, and can further obtain such instructions in a written form to include with the kit, and ship the kit and instructions to customer 140 (and providing input to billing department 124 that the product was shipped; or shipping department 119 can obtain from product storage 117 the kit for shipping, and can further provide the instructions to customer 140 in an electronic form, by accessing a database in company 100 that contains the instructions, and transmitting the instructions to customer 140 via the internet (not shown).

As further exemplified in FIG. 1, administration 120 includes order department 126, which receives input in the form of an order for a product from customer 140 or distributor 150. Order department 126 then provides output in the form of instructions to shipping department 119 to fill the order (i.e., to forward products as requested to customer 140 or distributor 150). Shipping department 119, in addition to filling the order, further provides input to billing department 124 in the form of confirmation of the products that have been shipped. Billing department 124 then can provide output in the form of a bill to customer 140 or distributor 150 as appropriate, and can further receive input that the bill has been paid, or, if no such input is received, can further provide output to customer 140 or distributor 150 that such payment may be delinquent. Additional optional component of company 100 include customer service department 122, which can receive input from customer 140 and can provide output in the form of feedback or information to customer 140. Furthermore, although not shown in FIG. 1, customer service 122 can receive input or provide output to any other component of company. For example, customer service department 122 can receive input from customer 140 indicating that an ordered product was not received, wherein customer service department 122 can provide output to shipping department 119 and/or order department 126 and/or billing department 124 regarding the missing product, thus providing a means to assure customer 140 satisfaction. Customer service department 122 also can receive input from customer 140 in the form of requested technical information, for example, for confirming that instructions of the invention can be applied to the particular need of customer 140, and can provide output to customer 140 in the form of a response to the requested technical information.

As such, the components of company 100 are suitably configured to communicate with each other to facilitate the transfer of materials and parts, devices, other components, products, and information within company 100, and company 100 is further suitably configured to receive input from or provide output to an outside party. For example, a physical path can be utilized to transfer products from product storage 117 to shipping department 119 upon receiving suitable input from order department 126. Order department 126, in comparison, can be linked electronically with other components within company 100, for example, by a communication network such as an intranet, and can be further configured to receive input, for example, from customer 140 by a telephone network, by mail or other carrier service, or via the internet. For electronic input and/or output, a direct electronic link such as a T1 line or a direct wireless connection also can be established, particularly within company 100 and, if desired, with distributor 150 or materials or parts 130 provider, or the like.

Although not illustrated, company 100 may contain one or more data collection systems, including, for example, a customer data collection system, which can be realized as a personal computer, a computer network, a personal digital assistant (PDA), an audio recording medium, a document in which written entries are made, any suitable device capable of receiving data, or any combination of the foregoing. Data collection systems can be used to gather data associated with a customer 140 or distributor 150, including, for example, a customer's shipping address and billing address, as well as more specific information such as the customer's ordering history and payment history, such data being useful, for example, to determine that a customer has made sufficient purchases to qualify for a discount on one or more future purchases.

Company 100 can utilize a number of software applications to provide components of company 100 with information or to provide a party outside of company access to one or more components of company 100, for example, access to order department 126 or customer service department 122. Such software applications can comprise a communication network such as the Internet, a local area network, or an intranet. For example, in an internet-based application, customer 140 can access a suitable web site and/or a web server that cooperates with order department 126 such that customer 140 can provide input in the form of an order to order department 126. In response, order department 126 can communicate with customer 140 to confirm that the order has been received, and can further communicate with shipping department 119, providing input that products such as a kit of the invention, which contains, for example, one or more cell matrix and instructions for use, should be shipped to customer 140. In this manner, the business of company 100 can proceed in an efficient manner.

In a networked arrangement, billing department 124 and shipping department 119, for example, can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor 150 can access a web site maintained by company 100 after establishing an online connection to the network, particularly to order department 126, and can provide input in the form of an order. If desired, a hard copy of an order placed with order department 126 can be printed from the web browser application resident at distributor 150.

The various software modules associated with the implementation of the present invention can be suitably loaded into the computer systems resident at company 100 and any party outside of company 100 as desired, or the software code can be stored on a computer-readable medium such as a floppy disk, magnetic tape, or an optical disk. In an online implementation, a server and web site maintained by company 100 can be configured to provide software downloads to remote users such as distributor 150, materials and parts 130, and the like. When implemented in software, the techniques of the present invention are carried out by code segments and instructions associated with the various process tasks described herein.

Accordingly, the present invention further includes methods for providing various aspects of a product (e.g., a kit and/or instructions of the invention), as well as information regarding various aspects of the invention, to parties such as the parties shown as customer 140 and distributor 150 in FIG. 1. Thus, methods for selling devices, products and methods of the invention to such parties are provided, as are methods related to those sales, including customer support, billing, product inventory management within the company, etc. Examples of such methods are shown in FIG. 1, including, for example, wherein materials and parts 130 can be acquired from a source outside of company 100 (e.g., a supplier) and used to prepare devices used in preparing a composition or practicing a method of the invention, for example, kits, which can be maintained as an inventory in product storage 117. It should be recognized that devices 112 can be sold directly to a customer and/or distributor (not shown), or can be combined with one or more other components 116, and sold to a customer and/or distributor as the combined product. The other components 116 can be obtained from a source outside of company 100 (materials and parts 130) or can be prepared within company 100 (materials and parts 114). As such, the term “product” is used generally herein to refer an item sent to a party outside of the company (a customer, a distributor, etc.) and includes items such as devices 112, which can be sent to a party alone or as a component of a kit or the like.

At the appropriate time, the product is removed from product storage 117, for example, by shipping department 119, and sent to a requesting party such as customer 140 or distributor 150. Typically, such shipping occurs in response to the party placing an order, which is then forwarded the within the organization as exemplified in FIG. 1, and results in the ordered product being sent to the party. Data regarding shipment of the product to the party is transmitted further within the organization, for example, from shipping department 119 to billing department 124, which, in turn, can transmit a bill to the party, either with the product, or at a time after the product has been sent. Further, a bill can be sent in instances where the party has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, etc.). Typically, billing department 124 also is responsible for processing payment(s) made by the party. It will be recognized that variations from the exemplified method can be utilized; for example, customer service department 122 can receive an order from the party, and transmit the order to shipping department 119 (not shown), thus serving the functions exemplified in FIG. 1 by order department 126 and the customer service department 122.

Methods of the invention also include providing technical service to parties using a product, particularly a kit of the invention. While such a function can be performed by individuals involved in product research and development, inquiries related to technical service generally are handled, routed, and/or directed by an administrative department of the organization (e.g., customer service department 122). Often communications related to technical service (e.g., solving problems related to use of the product or individual components of the product) require a two way exchange of information, as exemplified by arrows indicating pathways of communication between customer 150 and customer service department 122.

As mentioned above, any number of variations of the process exemplified in FIG. 1 are possible and within the scope of the invention. Accordingly, the invention includes methods (e.g., business methods) that involve (1) the production of products; (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products (e.g., one or more cell matrix); (c) storing the one or more first products of step (b); (d) combining the one or more first products of step (b) with one or more other components to form one or more second products (e.g., a kit); (e) storing the one or more first products of step (b) or one or more second products of step (d); (f) obtaining an order a first product of step (b) or a second product of step (d); (g) shipping either the first product of step (b) or the second product of step (d) to the party that placed the order of step (f); (h) tracking data regarding to the amount of money owed by the party to which the product is shipped in step (g); (i) sending a bill to the party to which the product is shipped in step (g); (j) obtaining payment for the product shipped in step (g) (generally, but not necessarily, the payment is made by the party to which the product was shipped in step (g); and (k) exchanging technical information between the organization and a party in possession of a product shipped in step (d) (typically, the party to which the product was shipped in step (g)).

The present invention also provides a system and method for providing information as to availability of a product (e.g., a device product, a kit product, and the like) to parties having potential interest in the availability of the kit product. Such a method of the invention, which encompasses a method of advertising to the general or a specified public, the availability of the product, particularly a product comprising instructions and/or a kit of the present invention, can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product.

Accordingly, the present invention provides methods for advertising and/or marketing devices, products, and/or methods of the invention, such methods providing the advantage of inducing and/or increasing the sales of such devices, products, and/or methods. For example, advertising and/or marketing methods of the invention include those in which technical specifications and/or descriptions of devices and/or products; methods of using the devices and/or products; and/or instructions for practicing the methods and/or using the devices and/or products are presented to potential interested parties, particularly potential purchasers of the product such as customers, distributors, and the like. In particular embodiments, the advertising and/or marketing methods involve presenting such information in a tangible form or in an intangible to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

The invention further provides methods associated with the design of custom products. These methods include, for example, (1) the taking an order from a customer for one or more cell matrix, (2) preparation of one or more cell matrix, (3) and providing (e.g., shipping) the support of (b) to the customer. Additionally, in particular embodiments, the customer may be billed for the support with the bill either being sent to the customer along with the support or sent separately.

FIG. 2 provides a schematic diagram of an information providing management system as encompassed within the present invention. In practice, the blocks in FIG. 2 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The information providing management system as shown in FIG. 2 is exemplified by company 200, which makes, purchases, or otherwise makes available devices and methods 210 that alone, or in combination, provide products 220, for example, instructions, devices and/or kits of the present invention, that company 200 wishes to sell to interested parties. To this end, product descriptions 230 are made, providing information that would lead potential users to believe that products 220 can be useful to user. In order to effect transfer of product descriptions 230 to the potential users, product descriptions 230 is provided to advertising agency 240, which can be an entity separate from company 200, or to advertising department 260, which can be an entity related to company 200, for example, a subsidiary. Based on the product descriptions 230, advertisement 250 is generated and is provided to media accessible to potential purchasers of products 260, whom may then contact company 200 to purchase products 220.

By way of example, product descriptions 230 can be in a tangible form such as written descriptions, which can be delivered (e.g., mailed, couriered, etc) to advertising agency 240 and/or advertising department 250, or can be in an intangible form such as entered into and stored in a database (e.g., on a computer, in an electronic media, etc.) and transmitted to advertising agency 240 and/or advertising department 250 over a telephone line, T1 line, wireless network, or the like. Similarly, advertisement 250 can be a tangible or intangible form such that it conveniently and effectively can be provided to potential parties of interest (e.g., potential purchasers of product 260). For example, advertisement 250 can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement 250 can be provided in the form of directed mailing of computer media containing the advertisement (e.g., CDs, DVDs, floppy discs, etc.) or of e mail (i.e., mail or e-mail that is sent only to selected parties, for example, parties known to members of an organization that includes or is likely to include potential users of products 220); of web pages (e.g., on a website provided by company 200, or having links to the company 200 website); or of pop-up or pop-under ads on web pages known to be visited by potential purchaser of products 260, and the like. Potential purchasers of products 260, upon being apprised of the availability of the products 220, for example, the kits of the present invention, then can contact company 200 and, if so desired, can order said products 220 for company 200 (see FIG. 1).

Other aspects and embodiments of the invention will be apparent to one or ordinary skill in the art in light of what is known in the art, in light of the following drawings and description of the invention, and in light of the claims.

### BRIEF DESCRIPTION OF THE FIGURES

FIG. 1 provides a schematic representation of a system for providing a product to a party such as a customer/purchaser.

FIG. 2 provides a schematic representation of a system for advising a party as to the availability of a product.

FIG. 3 shows growth of stem cell line BG01V in conditioned medium. using Matrigel™ or a stem cell matrix of the invention. At each time point shown, there is a set of two bars, with each bar representing the mean relative fluorescence (RFU) observed with the sample tested in the Alamar Blue test. The first bar on the left is for Matrigel™ and the second bar is for a stem cell matrix of the invention.

FIG. 4 shows results of experiments carried out to test the shelf-life of stem matrices and their use in combination with serum-free media. In this figure, the first bar on the left, for each set of the three bars shown at each time point, shows the Relative Fluorescence Unit (RFU) measured at the time point for the Matrigel™ Control. The second and third bars of each set of three bars shows the RFU for CELLstart™ ShelfLife sample and the CELLstart™ Control sample, respectively.

FIG. 5 shows the results of experiments carried out to test the multiple passage propagation of human mesenchymal stem cells in StemPro® MSC SFM. Panel A shows average net total cell number per T25 flask and Panel B shows the average population doubling time which was calculated for each condition (n=3). Seed density for cells grown in StemPro® MSC SFM and grown in DMEM+10% FBS was 10,000 cells/cm². The split frequency was every 3 days. Medium was changed every 2 days. The cell matrix used was CELLstart™ (1:100).

FIG. 6 shows the results of experiments carried out to differentiate mesenchymal stem cells cultured under serum-free conditions (StemPro® MSC SFM) using CELLstart™.

FIG. 7 shows the results of experiments carried out to test the growth rate of neural stem cells cultured under serum-free conditions (StemPro® NSC SFM) using CELLstart™.

FIG. 8 shows the results of experiments carried out to differentiate neural stem cells cultured grown under serum-free conditions using CELLstart™. Differentiation potential of NSC cultured in Stempro® NSC SFM. NSC cultured in Stempro NSC SFM differentiated to make neurons (A) and glial cells (B-C).

FIG. 9 shows human fetal fibroblasts cultured on 1:50 CELLstart™ coated T-225 flask in 15% Knockout™ SR XenoFree-supplemented media.

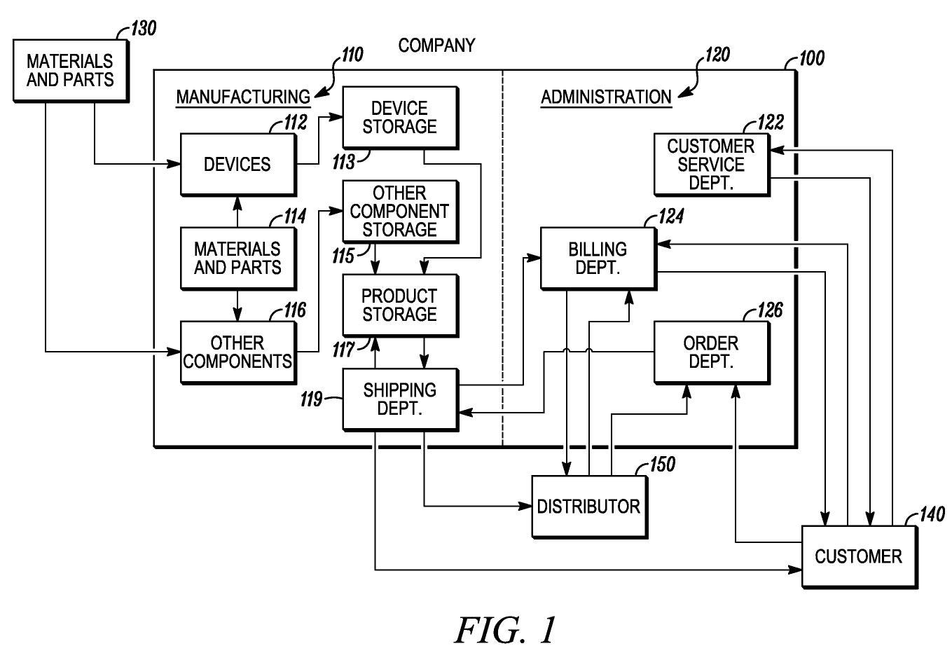

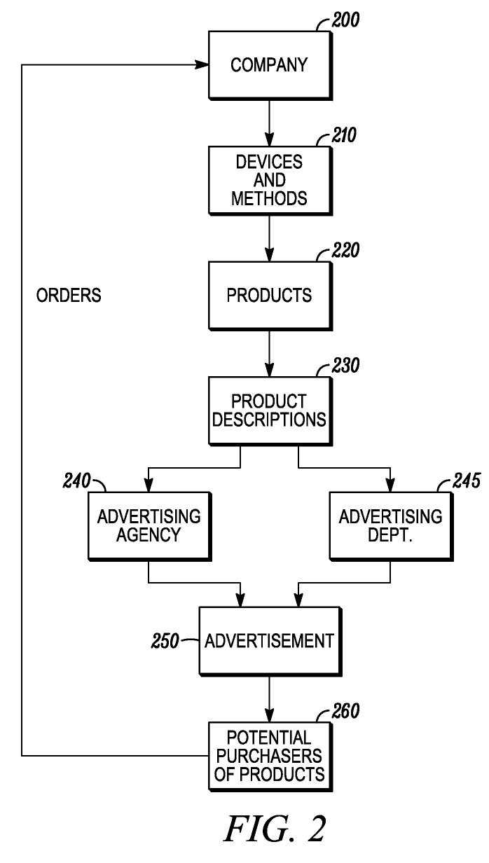

### DETAILED DESCRIPTION OF THE INVENTION

The present invention is drawn to compositions of matter and articles of manufacture that are or comprise novel cell matrices which may be cell matrices such as primary cell matrices and stem cell matrices that can be used for propagating cells which may be cells such as human primary cells, and embryonic stem cells and other adult stem cells, methods of manufacture and use thereof, and other methods related to the invention. More specifically, the invention relates to a novel cell matrix which may be a cell matrix such as a primary cell and stem cell matrix that consists essentially of Fibronectin and Albumin, and its use in combination with defined, serum-free and/or xeno-free culture media in cell cultures which may be cell cultures such as primary cell cultures or stem cell cultures, respectively.

Primary Cells

Cells that are cultured directly from a subject and before their subdivision and transfer to a sub culture are known as primary cells. To obtain primary cells, pieces of tissue from a host are placed in growth media and the cells which grow out this culture. With the exception of certain cells derived from tumors, most primary cell cultures have limited lifespan. After a certain number of population doublings, the cells undergo the process of senescence and stop dividing, while generally retaining viability. There are more than 200 primary cell types found in the mammalian body.

Of the many types of primary cell types found in the mammalian body, dermal keratinocytes, which include without limitation, human dermal keratinocytes. In some embodiments of the invention the primary cells in the articles of manufacture comprising the primary cells are fibroblast cells, which include without limitation, human fetal fibroblast cells. The source of primary cells can include without limitation mammals, including non-human primates and humans.

Primary cells can be grown in suspension or adherent cultures. Adherent cells require a surface, such as plastic, which may be coated with extracellular matrix components to increase adhesion properties and provide other signals needed for growth and differentiation. Most cells derived from solid tissues are adherent.

Of the more than 200 primary cell types found in the mammalian body, dermal keratinocytes, which include without limitation, human dermal keratinocytes and fibroblast cells, which include without limitation, human fetal fibroblast cells, can be grown in adherent cultures. The source of these primary cells can include without limitation mammals, including non-human primates and humans.

Stem Cells

Stem cells are a unique type of cell having many potential uses. Unlike most mammalian cells, stem cells have the ability to renew themselves through mitotic cell division and can differentiate into a wide variety of specialized cell types. In a developing embryo, stem cells can differentiate into any of the specialized embryonic tissues; in adult organisms, stem cells and progenitor cells act as a source of new cells for the body, replenishing or repairing specialized cells. Three broad categories of mammalian stem cells are known: embryonic stem cells, derived from blastocysts; adult stem cells, which are found in adult tissues; and cord blood stem cells, which are found in the umbilical cord.

Embryonic stem cells (ES cells, ESCs) are derived from the epiblast tissue of the inner cell mass (ICM) of a early stage embryo such as a blastocyst or an earlier morula stage embryo. ESCs are pluripotent, giving rise during development to all derivatives of the three primary germ layers: ectoderm, endoderm and mesoderm. Ideally, they can develop into each of the more than 200 cell types of the adult body if properly stimulated.

Embryonic stem cells have been produced from a variety of non-human primates. Non-limiting examples include stem cells from Macaca mulatta (rhesus macaque, see Thomson et al., Proc. Natl. Acad. Sci. U.S.A. 92:7844-7848, 1995), Macaca fascicularis (long-tailed macaque, see Vrana et al., Proc. Natl. Acad. Sci. U.S.A 100:11911-1191, 2003), and Callithrix jacchus (common marmoset, see Thomson and Marshall, Curr. Top. Dev. Biol. 38:133-165, 1998).

Human embryonic stem (hES) cells are known in the art and can be prepared in various manners, including as non-limiting examples those described by Thomson et al. (U.S. Pat. No. 5,843,780; Science 282:1145, 1998). For example, human blastocysts can be derived from in vivo preimplantation embryos, or in vitro fertilized (IVF) embryos can be used, or one or more cell human embryos can be expanded to the blastocyst stage (Bongso et al., Hum. Reprod. 4:706-713, 1989; Gardner et al., Fertil. Steril. 69:84-88, 1998). Embryonic stem cell-like cells can also be produced by transfection of certain stem cell genes into non-pluripotent cells, such as adult fibroblasts. Stem cells made in this manner are referred to as induced pluripotent stem cells. The transfection has been reported to be achieved through the use of a retroviral vector containing the genes, Oct3/4, Sox2, Klf4 and c-Myc (Takahashi K et al., Cell 131(5): 861-872, 2007) and through the use of a lentivirual vector containing the genes, OCT4, SOX2, NANOG and LIN28 (Yu J, Vodyanik M A et al., Science 318(5858): 1971-1920, 2007). Many techniques useful in stem cell culture and research are known in the art; some are disclosed in: Embryonic Stem Cells Methods and Protocols (Methods in Molecular Biology Vol. 185), Turksen, Ed., Human Press, Totawa, N.J., 2002.

A significant limitation to the use of stem cells is that initially they were cultured in a way that is dependent on the use of mouse cells (MEFs) as a feeder layer. The use of feeder layers raises several concerns: the potential presence of xenogeneic contaminants which might, for example, restrict transplantation of hES cells to humans; the potential for variability in MEFs from batch-to-batch and laboratory-to-laboratory, which may contribute to some of the variability in experimental results; and the use of a second population of cells (the feeder layer), which increases the workload and subsequently limits the large-scale culture of human ES cells.

Accordingly, a goal in stem cell technology has been the development of feeder-free cultures. Over time, researchers have come to understand that necessary components stem cell culture include, in addition to culture media, various uncharacterized soluble factors that promote stem cell growth but suppress (or at least do not induce) differentiation, and a matrix for stem cell attachment. In stem cell culture, the stem cell matrix is coated onto a substrate, such as portion of the interior of a culture vessel that also contains media, soluble factors and stem cells.

Stem Cell Matrices

The current stem cell matrix of choice is often Matrigel™, which is an extracellular matrix extracted from murine Engelbreth-Holm-Swarm tumor cells having a “complex and variable composition” (Dailey, Blood 105:4550, 2005) having some degree of lot-to-lot variation. The major component of Matrigel™ is laminin, followed by collagen IV, heparan sulfate proteoglycans, enactin, and nidogen (Kleinman et al., Biochemistry: 21: 6188-6193, 1982). Matrigel™ also comprises TGF-beta, fibroblast growth factor, tissue plasminogen activator (McGuire and Seeds, J. Cell. Biochem. 40:215-227, 1989) and other factors that are produced in the EHS tumor. Although it has been studied and characterized to some degree, Matrigel™ is not a defined formulation.

Besides Matrigel™, other stem cell matrices that have been used include without limitation those listed below, as well as others. For reviews and summaries, see Mallon et al., Int. J. Biochem. Cell Biol. 38:1063-75, 2006; and Skottman and Hovatta, Reproduction 132:691-698, 2006.

The use of fibronectin as a stem cell matrix has been described under limited conditions and in the presence of a complex variety of other factors (20% serum replacement and a combination of growth factors: TGF-beta1, leukemia inhibitory factor, basic fibroblast growth factor) has been described (Amit et al., Biol Reprod: 70: 837-845, 2004).

The use of human serum as a stem cell matrix has been described by Stojkovic et al. (Stem Cells 23:895-902, 2005) and Ellerström et al. (Stem Cells 24:2170-2176, 2006).

The use of laminin as a stem cell matrix has been described (Beattie et al., Stem Cells: 23: 489-495, 2005).

The use of porous substrates such as alginate matrix as a stem cell matrix has been described (Gerecht-Nir et al., Biotechnol Bioeng: 88: 313-320, 2004).

The use of hyaluronic acid hydrogel as a stem cell matrix has been described (Gerecht et al., Proc. Natl. Acad. Sci. U.S.A 104:11298-11303, 2007).

The use of three-dimensional nanofibrillar surfaces as a stem cell matrix has been described (Nur-E-Kamal et al., Stem Cells 24:426-433, 2006).

Fibronectin

Fibronectin is a connective tissue glycoprotein that consists of two structurally similar (but not necessarily identical) disulfide-bonded subunits (Schwarzbauer et al., Proc. Natl. Acad. Sci. USA, Vol. 82, pp. 1424-1428, March 1985). There are two principle types of fibronectin: (a) plasma fibronectin, which appears to be produced by hepatocytes (Tamkun and Hynes, J. Biol. Chem., Vol. 258, Issue 7, 4641-4647, 04, 1983, 1983) and is found in the circulation, and (b) cellular fibronectin, which is synthesized by a variety of cell types. For a review, see Goodheart and Silverman, SIM Industrial Microbiology News 41:266-272. Both types of fibronectin can be found in a soluble form in blood, other body fluids, and culture medium, and in an insoluble, usually fibrillar, form in the extracellular space of connective tissues, basement membranes, and cultured cells.

Although cellular and plasma fibronectins have many striking similarities, the locations of the polypeptide chain differences between these two proteins indicate that plasma fibronectin cannot be derived from cellular fibronectin by means of simple post-translational proteolysis. Instead, these different types of fibronectin arise from alternative splicing of a single fibronectin gene transcript.

In humans, alternative splicing at three different domains can produce up to 20 fibronectin subunit isoforms (Kornblihtt et al., Nucleic Acids Res. 12:5853-5868, 1984; Kornblihtt et al., The EMBO Journal 4:1755-1759, 1985; Borsi et al., FEBS Lett. 261:175-178, 1990; Kornblihtt et al., FASEB. J. 10:248-257, 1996).

Cellular fibronectin isoforms have a distinctive protein domain, named “extra domain I (ED I)”, that is absent from serum fibronectin. Serum fibronectin isoforms comprise a protein domain, named “the extra domain II (ED II)”, that is not present in cellular fibronectin. As used in the context of the invention, the term “serum fibronectin” refers to any fibronectin molecule that lacks ED I. In some embodiments, the term “serum fibronectin” refers more specifically to fibronectin molecules that lack ED I but comprise ED II.

Fibronectins have a relatively simple structure and consist of a dimer of two subunits joined by disulfide bonds near the COOH termini (Hynes, R. O. (1990). Fibronectins. Springer-Verlag, New York). Three types of homologous repeating units (referred to as types I, II and III domains/repeats) form each subunit. Alternative splicing of a single primary transcript leads to the inclusion or exclusion of three domains in the central part of the molecule.

The amino acid sequence of human plasma fibronectin was initially determined via polypeptide digestion and analysis (Garcia-Pardo et al., J. Biol. Chem. 258:12670-32674, 1983; Garcia-Pardo et al., J. Biol. Chem. 26010320-10325, 1985; Garcia-Pardo et al., Biochem. J. 241, 923-928, 1987). The nucleotide sequence of the human fibronectin gene is also known (Kornblihtt et al., Nucleic Acids Res. 12:5853-5868, 1984; Kornblihtt et al., The EMBO Journal 4:1755-1759, 1985).

Genetic variations (polymorphisms) in the gene for fibronectin are known (see, e.g., Siemianowicz et al., Oncology Reports 8:1289-1292, 2001). When practicing the invention with regards to stem cells being produced for use in or for production of therapeutic molecules for any given individual, it may be preferable to use a fibronectin having the same polymorphisms as that individual. Such variant fibronectins can be produced via recombinant DNA technology by those skilled in the art.

Producing fibronectin via recombinant DNA technology should result in production of protein lots that can be somewhat more homogenous than protein isolated from biological sources. As regards a protein preparation, the term “more homogenous” (synonymous with “less heterogeneous”) signifies that the range of molecular weights of the protein is smaller because by way of non-limiting example, fewer less than full-length (truncated) forms of the protein are present, post-translation modification (e.g., glycosylation) is more consistent or is eliminated, etc. Recombinant human fibronectins, including human plasma fibronectin, have been described (see Dufour et al., Exp. Cell Res. 193:331-338, 1991; Akamatsu et al., Cancer Res. 56:4541-4546, 1996).

Albumin

Albumin is an umbrella term for a type of protein which is soluble in pure water, precipitable from solution by strong acids, and coagulable by heat in acid or neutral solution. Numerous types of albumin are widely distributed throughout the tissues and fluids of plants and animals, and two of the most familiar examples of albumin can be found in egg whites and in human blood. Within the human body, albumin transports essential fatty acids from adipose tissue to muscle tissue. It also contributes to the regulation of osmosis, helping to transport hormones, drugs, and other substances through the blood.

Serum albumin is the most abundant blood plasma protein and is produced in the liver and forms a large proportion of all plasma protein. The human version is human serum albumin (HSA), and it normally constitutes about 60% of human plasma protein; all other proteins present in blood plasma are referred to collectively as globulins.

In humans, albumin is the most abundant plasma protein, accounting for 55-60% of the measured serum protein. It is produced in the liver and the concentration in serum is 35-50 mg/ml. It consists of a single polypeptide chain of 585 amino acids with a molecular weight of 66,500 Da. The mature, circulating molecule is arranged in a series of alpha helices, folded and held by 17 disulphide bridges, and is characterized by having no carbohydrate moiety, a scarcity of tryptophan and methionine residues, and an abundance of charged residues, such as lysine, arginine, glutamic acid and aspartic acid (for a review, see Peters T J. The albumin molecule: its structure and chemical properties. In: All About Albumin. Biochemistry, Genetics And Medical Applications. San Diego: Academic Press, 1996: 9-75).

The amino acid sequence of human albumin was initially determined via polypeptide digestion and analysis (Meloun et al., FEBS Letters 58:134-137, 1975; Walker, Eur. J. Biochem. 69:517-526, 1976). The nucleotide sequence of the gene encoding HSA has been determined (Lawn et al., Nucleic Acids Res. 9:6103-6114, 1981; Dugaiczyk et al., Proc. Natl. Acad. Sci. USA 79:71-75, 1982; Minghetti et al., J. Biol. Chem. 261:6747-6757, 1986).

Genetic variations (polymorphisms) in the gene for HSA are known (see, e.g., Minghetti et al., J. Biol. Chem. 261:6747-6757, 1986; Dugaiczyk et al., Proc. Natl. Acad. Sci. USA 79:71-75, 1982; Lawn et al., Nucleic Acids Res. 9:6103-6114, 1981; Mariotti et al., Protides Biol. Fluids Proc. Colloq. 33, 177-179, 1985; Murray et al., Proc. Natl. Acad. Sci. USA 81:3486-3490, 1984; and Lavareda et al., Hum. Genet. 67:48-51, 1984). When practicing the invention with regards to cells which may be cells such as primary cells or stem cells being produced for use in or for production of therapeutic molecules for any given individual, it may be preferable to use an albumin having the same polymorphisms as that individual. Such variant albumins can be produced via recombinant DNA technology by those skilled in the art.

Producing albumin via recombinant DNA technology should result in production of protein lots that can be somewhat more homogenous than protein isolated from biological sources. Recombinant human serum albumin (HSA) has been described (see, e.g., EP 330 451 and EP 361 991; Lawn et al., Nucleic Acids Res. 9:6103-6114, 1981; Latta et al., Bio/Technology 5:1309-1314, 1987; Dodsworth et al., Biotechnol. Appl. Biochem. 24:171-176, 1996; Saunders et al., J. Bacteriol. 169:2917-2925, 1987; Etcheverry et al., Biotechnology 4:726-730, 1986; Sleep et al., Biotechnology 8:42-46, 1990; Cousens et al., Nucleic Acids Res. 18:1308, 1990; Sijmons et al., Biotechnology 8:217-221, 1990). Recombumin® (Novozymes Biopharma AB, Lund, Sweden) is a genetically engineered protein expressed in yeast cells (Saccharomyces cerevisiae) that is commercially available.

Cell Culture Media

Cell culture media provide the nutrients necessary to maintain and grow cells in a controlled, artificial and in vitro environment. The characteristics and compositions of the cell culture media vary depending on the particular cellular requirements. Important parameters include osmolarity, pH, and nutrient formulations.

Media formulations have been used to cultivate a number of cell types including animal, plant and bacterial cells. Cells cultivated in culture media catabolize available nutrients and produce useful biological substances such us monoclonal antibodies, hormones, growth factors and the like. Such products have therapeutic applications and, with the advent of recombinant DNA technology, cells can he engineered to produce large quantities of these products. Cultured cells are also routinely used for the isolation, identification and growth of viruses. Thus, the ability to cultivate cells in vitro is not only important for the study of cell physiology, but is also necessary for the production of useful substances which may not otherwise be obtained by cost-effective means.

Cell culture media formulations are well documented in the literature, and a number of media are commercially available. In early cell culture work, media formulations were based on the chemical composition and physiochemical properties (e.g., osmolality, pH, etc.) of blood and were referred to as “physiological solutions” (Ringer, J. Physiol 3:380-393, 1880; Waymouth, In: Cells and Tissues in Culture. Vol. 1, Academic Press, London, pp. 99-142, 1965; and Waymouth, In Vitro 6:109-127, 1970). However, cells in different tissues of the mammalian body are exposed to different microenvironments with respect to oxygen/carbon dioxide partial pressure and concentrations or nutrients, vitamins, and trace elements. Accordingly, successful in vitro culture of different cell types often require the use of different media formulations. Typical components of cell culture media include amino acids, organic and inorganic salts, vitamins, trace metals, sugars, lipids and nucleic acids, the types and amounts of which may vary depending upon the particular requirements of a given cell or tissue type.

Typically, cell culture media formulations are supplemented with a range of additives, including undefined components such as fetal bovine serum (FBS) (10-20% v/v) or extracts from animal embryos, organs or glands (0.5-10% v/v). While FBS is probably the most commonly applied supplement in animal cell culture media, other serum sources are also routinely used, including newborn calf, horse and human. These types of chemically undefined supplements serve several useful functions in cell culture media (Lambert et al., In: Animal Cell Biotechnology, Vol. 1, Spier et al., Eds., Academic Press New York, pp. 85-122, 1985). For example, these supplements provide carriers or chelators for labile or water-insoluble nutrients; bind and neutralize toxic moieties; provide hormones and growth factors, protease inhibitors and essential, often unidentified or undefined low molecular weight, nutrients; and protect cells from physical stress and damage. Thus, serum and/or animal extracts are commonly used as relatively low-cost supplements to provide an optimal culture medium for the cultivation of animal cells.

Unfortunately, the use of serum or animal extract in tissue culture applications has several drawbacks (Lambert, et al., 1985). For example, the chemical composition of these supplements may vary between lots, even from a single manufacturer. The supplements of animal or human origin may also be contaminated with infectious agents (e.g. mycoplasma and viruses) which can seriously undermine the health of the cultured cells when those contaminated supplements are used in cell culture media formulations and may additionally pose a health risk in cell therapy and other clinical applications. Cell surface chemistry, which is a critical portion of the in vitro microenvironment for many cell types, can be adversely modified via adsorption or incorporation of serum or extract proteins. In the industrial production of biological substances, serum and animal extract supplementation of culture media can also complicate and increase the costs of purification of the desired substances from the culture media due to nonspecific co-purification of serum or extract proteins.

The use of undefined components such as serum or animal extracts also prevent the true definition and elucidation of the nutritional and hormonal requirements of the cultured cell, thus eliminating the ability to study, in a controlled way, the effect of specific growth factors or nutrients on cell growth and differentiation in culture. Moreover, undefined supplements prevent the researcher from studying aberrant growth and differentiation and the disease-related changes in cultured cells.

To overcome these drawbacks of the use of serum or organ/gland extracts, a number of defined media have been developed. These media, which often are specifically formulated to support the culture of a single cell type, are completely defined (i.e., they contain no undefined components) and comprise defined quantities of purified growth factors, proteins, lipoproteins and other substances in lieu of those usually provided by the serum or extract supplement. Since the components (and concentrations thereof) in such culture media are precisely known, these media are generally referred to as “defined culture media.”

A term related to defined media is “serum-free media” or “SFM.” A number of SFM formulations are commercially available, such as those designed to support the culture of endothelial cells, keratinocytes, monocytes/macrophages, fibroblasts, chondrocytes or hepatocytes which are available from GIBCO/Invitrogen (Carlsbad, Calif., US).

The distinction between serum-free and completely defined media is that some SFM, although devoid of serum, comprise other uncharacterized components such as biological extracts. Examples of SFM that have been reported or that are available commercially that contain uncharacterized components include by way of non-limiting example several formulations supporting in vitro culture of keratinocytes (Boyce et al., Invest. Dermatol. 81:33-40, 1983; Wille et al., J. Cell Physiol. 121:31-44, 1984; Pittelkow and Scott, Mayo Clin. Proc. 61:771-777, 1986; Pirisi et al., J. Virol. 61:1061-1066, 1987; Shipley and Pittelkow, Arch. Dermatol. 123:1541-1544, 1987; Shipley et al., J. Cell. Physiol 138:511-518, 1989; Daley et al., FOCUS (GIBCO/Invitrogen) 12:68--, 1990; and U.S. Pat. Nos. 4,673,649 and 4,940,666). SFM that include uncharacterized components are not completely defined.

Defined media generally provide several distinct advantages to the user. For example, the use of defined media facilitates the investigation of the effects of a specific growth factor or other medium component on cellular physiology, which may be masked when the cells are cultivated in serum- or extract-containing media. In addition, defined media typically contain much lower quantities of protein (indeed, defined media are often termed “low protein media”) than those containing serum or extracts, rendering purification of biological substances produced by cells cultured in defined media far simpler and more cost-effective.

Some extremely simple defined media, which comprise or consist essentially of vitamins, amino acids, organic and inorganic salts and buffers have been used for cell culture. Such media (often called “basal media”), however, are usually seriously deficient in the nutritional content required by most animal cells. Accordingly, most defined media incorporate into the basal media additional components to make the media more nutritionally complex, but to maintain the serum-free and low protein content of the media. Examples of such components include serum albumin from bovine (BSA) or human (HSA); certain growth factors derived from natural (animal) or recombinant sources such as EGF or FGF; lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth in: Cell Culture Methods for Molecular and Cell Biology, Vol. 1: Methods for Preparation of Media, Supplements, and Substrata for Serum-Free Animal Cell Culture, Barnes et al., eds., New York: Alan R. Liss, Inc., pp. 23-68, 1984, and by Gospodarowicz, Id., at pp. 69-86, 1984).

Stem Cell Culture Media

In addition to feeder cells, another way to provide or supplement the soluble factors is to use conditioned media, i.e., media that has been exposed to feeder cells. Typically, to date, cultures are generally expanded either on murine embryonic feeder (MEF) layers or in “conditioned” medium on a matrix (typically Matrigel™) in the absence of MEF. Moreover, serum, another undesired contaminant, is often also added to the media as a supplement in such cultures.

The ultimate goal of these efforts is a set of ES cell culture components—media, matrix, and soluble factors—wherein each of these components (1) is completely defined, (2) comprises a minimum number of molecular components, (3) is serum-free, and (4) is xeno-free.

Rajala et al. (Human Reproduction 22:1231-1238, 2007) describe testing nine different xeno-free culture media for human embryonic stem cell cultures. None of the studied xeno-free media were able to maintain the undifferentiated growth of hESC, although some media containing 20% human serum was found to sustain undifferentiated hESC proliferation to a limited extent.

Ellerström et al. (Stem Cells 24: 2170-2176, 2006) describe a xeno-free medium supplemented with human serum, which supports long-term (>50 passages) culture of hESCs in an undifferentiated state.

Lu et al. (Proc. Natl. Acad. Sci. U.S.A 103: 5688-5693, 2006) describe “a simple medium [termed hESC Cocktail (HESCO)] containing basic fibroblast growth factor, Wnt3a, April (a proliferation-inducing ligand) BAFF (B cell-activating factor belonging to TNF), albumin, cholesterol, insulin, and transferrin, which is sufficient for hESC self-renewal and proliferation.”

Conclusion

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

Although the foregoing invention has been described in some detail by way of illustration and example for purposes of clarity of understanding, it will be obvious that certain changes and modifications may be practiced within the scope of the appended claims.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The examples that follow are provided by way of further illustration, and are not meant to imply any limitation to practice of the claimed invention.

### Example 1

### Preparation of Exemplary Matrix Solutions

In a total volume of 60 ml, 4.8 ml (1.2 g) of a 25% Human Serum Albumin solution is added to 55.2 ml of a 1 mg/ml Human Plasma Fibronectin solution.

The 25% Human Serum Albumin (HSA) used is from ABO Pharmaceuticals catalog no. 1500233.

The 1 mg/ml Human Plasma Fibronectin (FN) solution used is Calbiochem, catalog no. 970458.

The resulting stock formulation comprises 0.92 mg/ml Human Plasma Fibronectin and 2% Human Serum Albumin. A shorthand description of this solution is “1 mg/ml FN and 2% HSA”.

The stock solution is diluted 1:50 in Dulbecco's Phosphate-Buffered Saline (D-PBS) with calcium, magnesium and phenol red (Invitrogen, Carlsbad, Calif., US, catalog #14040) to yield a solution comprising 0.02 mg/ml Human Plasma Fibronectin and 0.04% Human Serum Albumin. The dilute solution is used to coat surfaces such as the floors of Petri dishes and multiwell titer plates that are used to culture cells.

Solutions are stable for 19 months stored at from about 2° C. to about 8° C. or frozen for long-term storage.

### Example 2

### Instructions for Preparing Matrix-Coated Plates

1. Prepare a dilute solution of CELLstart™ (prepared according to Example 1) at room temperature, from about 15° C. to 30° C.

2. Pipette the dilute solution of CELLstart™ onto the plate at a volume of 0.078 ml/cm².

3. Once added to the plate, incubate the plates with the coating solution at about 37° C. in a 5% CO₂ incubator for from about 60 to about 120 minutes.

4. After incubation, decant or aspirate the coating solution out of the wells.

5. Culture media and stem cells can be added at this point, or the plate can kept “wet” or dried and packaged for storage, transport and later use.

6. Preparing “wet” plates: Add from about 0.078 ml/cm² of a dilute CELLstart™ solution to each well. You may need to add a seal to the top of the plate with an impermeable membrane and store at from about 2° C. to about 8° C. protected from light.

7. Preparing “dry” plates: Dry the plate under sterile conditions by letting it sit in the laminar flow hood from about 1 to about 24 hours, protected from. When dry, seal the plate in an air-tight foil pouch with a desiccant and store at from about 2° C. to about 8° C. protected from light.

### Example 3

### Stem Cell Culture with Conditioned Media

3.1 Procedure

A series of 96-well plates is coated with a matrix solution of the invention or a control matrix (Matrigel™). Human embryonic stem cells (cell line BG01V) are grown in the 96-well plates for 4 days after a ˜1:4 split. Initial setup contains 100 uL of medium with cells diluted ˜1:4. Each day, cells are fluid-changed (old media is removed by aspiration and replaced with 100 uL of fresh media). The medium in these experiments is mouse embryonic fibroblast conditioned medium (MEFCM).

Cell viability (CV) is measured as follows. On day 4, 15 uL of Alamar Blue™ (United States Biological, Swampscott, Mass.) is added to each well and the plates are read on a fluorescent plate reader over an incubation period of 6 hours. Alamar Blue™ (BUF012a) is an aqueous dye that is used to quantitatively measure cell viability and cell proliferation. Alamar blue measures metabolic activity by change in fluorescence (Ahmed et al., J. Immunol. Methods 170:211-224, 1994). Measurements (in RFU, mean relative fluorescence units) are taken at t=1 hr., 2.25 hr., 3.25 hr., 4.25 hr., 5.25 hr. and 6 hr. For Matrigel™, n=48 wells; for CELLstart™ (“HSA/FN”), n=32 wells.

The CV % is calculated as:

(Standard Deviation/Mean)×100

A low CV % (less than about 20%, preferably less than about 10%, most preferably less than about 8%) indicates that cellular growth is not deviating too far from the average and that there is not too much variability within the assay.

3.2 Results

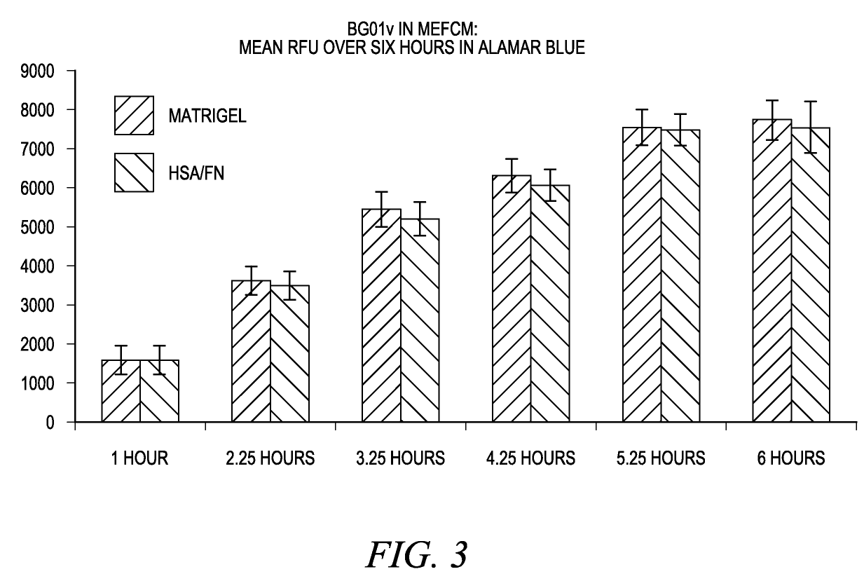

The results (FIG. 3) show that there is a steady increase in Alamar Blue™ readout over 6 hours, indicating that cell growth is occurring. The rate of growth is comparable in cells cultured on either Matrigel™ or CELLstart™ matrices. CV % s are low, and average around 6-7% at 4 hours, the preferable time of read.

3.3 Conclusion

When used in combination with conditioned media, the CELLstart™ matrices of the invention, consisting essentially of Fibronectin and Albumin, work as well as Matrigel™ to support attachment and growth of human embryonic stem cells.

### Example 4

### Stem Cell Culture with Defined, Unconditioned, and Serum-Free Media (Shelf-Life Studies)

4.1 Procedure

BG01V cells are grown in a series of 96-well plates coated with 2 different matrix solutions for 3 days after a ˜1:4 split. The 2 different matrix solutions are:

(1) “CELLstart™ Control” is a freshly-prepared 1:50 dilution of a stock CELLstart™ solution that is produced under a cGMP environment. The stock solution is composed of 0.92 mg/ml Fibronectin and 2% HSA prepared according to Example 1.

(2) “CELLstart™ ShelfLife” is a stock solution of 0.92 mg/ml Fibronectin and 2% HSA that is stored at 2° C. to 8° C. for 13 months and is prepared according to Example 1

“Matrigel 1:200 Control” is a freshly diluted (200 fold) preparation of Matrigel™ that is stored for the same period of time and under the same conditions as the preceding “CELLstart™ ShelfLife” and “CELLstart™ Control samples.

Initial setup contains 200 uL of serum-free medium with cells diluted ˜1:350. On day 2 and 3, fluid is removed by aspiration and replaced with 150 uL of serum-free media (STEMPro™ hESC SFM; Invitrogen, Carlsbad, Calif., US).

On day 3, 15 uL of Alamar Blue™ is added to each well and the plates are read on a fluorescent plate reader over an incubation period of 8 hours. For all samples to test, n=24.

4.2 results

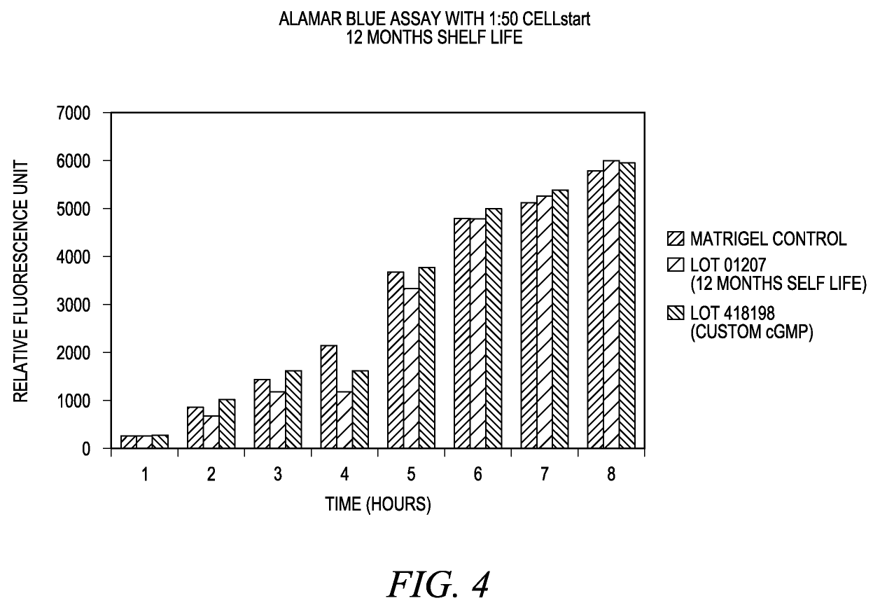

The results (FIG. 4) show that there is a steady increase in Alamar Blue™ readout over the 8 hour incubation period. Cell growth is comparable in all conditions tested.

4.3 Conclusions

(A) When used in combination with defined, unconditioned and serum-free media, the CELLstart™ matrices of the invention, consisting essentially of Fibronectin and Albumin, work as well as Matrigel™ to support attachment and growth of human embryonic stem cells.

(B) The CELLstart™ matrices of the invention, consisting essentially of Fibronectin and Albumin, have a shelf life of at least about 12 months when stored at 2° C. to 8° C.

Further experiments demonstrate a shelf-life of at least about 18 months when stored at 2° C. to 8° C.

### Example 5

### Titration Studies

5.1 Procedure

The following CELLstart™ solutions is prepared by diluting the 1× and 10× solutions of Example 1 to the different concentrations of fibronectin and human serum albumin using Dulbecco's Phosphate-Buffered Saline (DPBS) with calcium and magnesium.

TABLE 1
Formulations To Use in Titration Studies

| Protein | Theoretical Total Protein |
|---|---|
| Concentrations | Conc. (mg/ml) |
| 0.4 mg/ml fibronectin, 0.8% HSA | 8.4 |
| 0.04 mg/ml fibronectin, 0.08% HSA | 0.84 |
| 0.01 mg/ml fibronectin, 0.02% HSA | 0.21 |
| 0.004 mg/ml fibronectin, 0.008% HSA | 0.084 |
| 0.001 mg/ml fibronectin, 0.001% HSA | 0.021 |
| BD Bioscience Matrigel ™, 1:200 Control | — |

To a series of 35 mm tissue culture Petri dishes, a volume (typically, about 1 mL) of each dilution just sufficient to cover the surface of the dish is added, and the dishes are incubated in a 37° C., 5% CO₂ incubator for 60 to 120 minutes.

Following the incubation step, the CELLstart™ solution is carefully removed by aspiration without rinsing. BG01 V cells are then added to the dish. The cells are added suspended in a serum-free media that can be used to grow hESC (StemPro® hESC SFM; Invitrogen, Carlsbad, Calif., US).

Cells are cultured for 3 passages in duplicate 35 mm tissue culture Petri dishes precoated with CELLstart™ at the different protein concentrations as listed above.

Pictures of BG01V cells on passage 3, day 4 in duplicate 35 mm tissue culture Petri dishes are taken and used to evaluate cell colony morphology. Alkaline Phosphatase, produced by undifferentiated cells such as stem cells, is measured.

5.2. Results

TABLE 2
Results of Titration Studies

| | Supports BG01V Cell |
|---|---|
| Matrix | Culture? |
| 0.4 mg/ml Fibronectin | YES |
| with 0.8% HSA | |
| 0.04 mg/ml Fibronectin | YES |
| with 0.08% HSA | |
| 0.01 mg/ml Fibronectin | YES |
| with 0.02% HSA | |
| 0.004 mg/ml Fibronectin | NO* |
| with 0.008% HSA | |
| 0.001 mg/ml Fibronectin | NO* |
| with 0.002% HSA | |
| BD Bioscience Matrigel ™ 1:200 Control | YES |

*Dishes precoated with either 0.004 mg/ml Fibronectin with 0.008% HSA or 0.001 mg/ml Fibronectin with 0.002% HSA do not support robust undifferentiated stem cell growth. Most of the colonies in these dishes show no attachment and/or differentiated colonies. Alkaline phosphatase activity is as expected for stem cells.

5.3 Conclusion

Acceptable concentrations of stem cell matrices range from about 0.01 mg/ml Fibronectin with about 0.02% HSA to about 1 mg/mL Fibronectin with about 2% HSA. Under these conditions, the former set of concentrations is the lowest protein concentration that will support human embryonic stem cell attachment, growth, and expansion while maintaining characteristics of undifferentiated cell morphologies. The formulations with the next lowest concentrations (0.004 mg/ml Fibronectin and 0.008% HSA) are inadequate under these conditions.

### Example 6

### Mesenchymal Stem Cells (MSC) Growth Under Serum-Free Conditions

6.1 Procedures.

CELLstart™ with 0.92 mg/ml fibronectin and 20 mg/ml of HSA is diluted 1:100 in Dulbecco's Phosphate Buffered Saline. 5 ml of the dilute solution was used to coat a T25 tissue culture flask by incubating in a 37° C., 5% CO₂ incubator for 60 to 120 minutes.

Following the incubation step, the CELLstart™ solution was carefully removed by aspiration without rinsing. Mesenchymal stem cells are seeded to each T25 flask to provide 1×10⁴ cells/cm². Flasks were mixed and swirl to ensure even distribution of cells inside the flasks.

Cells were incubated in a humidified 37° C., 5% CO₂ incubator and fluid changed every 2 days with fresh, pre-warmed complete StemPro® MSC SFM (Invitrogen, Carlsbad, Calif., US). Cells were passaged upon reaching desired level of confluence, 80%.

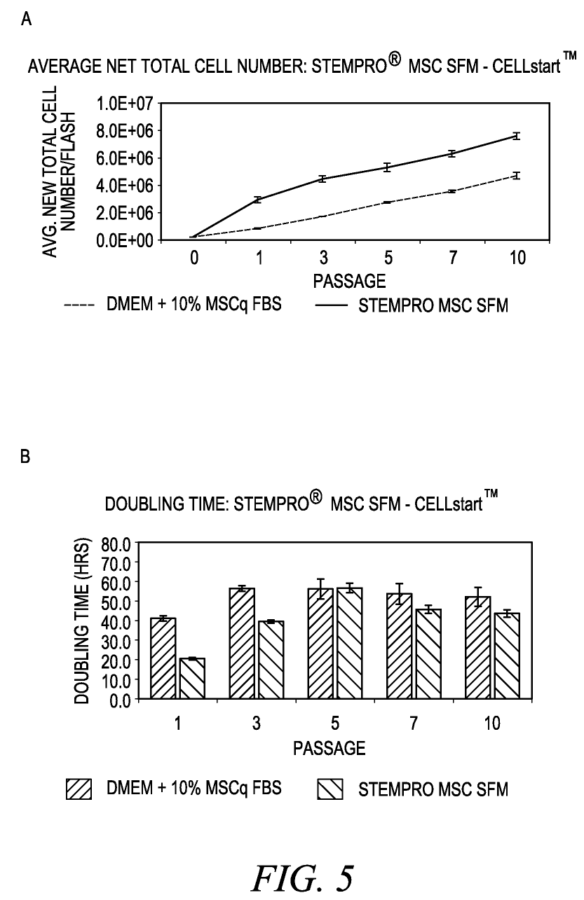

FIG. 5 shows the results of this growth rate experiment.

Mesenchymal stem cells cultured in StemPro® MSC SFM and CELLstart™ coated tissue culture containers were differentiated to prove these cells maintained tri-lineage potential when cultured in this condition. Cells were differentiated into chondrocytes, osteocytes, and adipocytes.

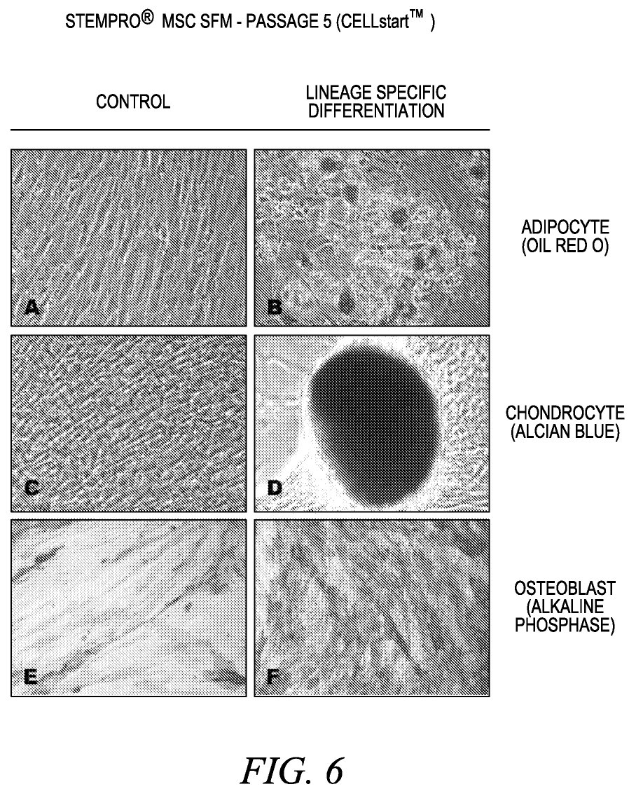

FIG. 6 show the results of this mesenchymal stem cell differentiation study.

6.2 Conclusion

Mesenchymal stem cells cultured on CELLstart™ coated dishes in a serum-free medium, StemPro® MSC SFM, were able grow, expand, and maintain tri-lineage differentiation potential into adipocytes, chondrocytes, and osteoblasts. This is evidenced by data showing faster growth rate in comparison to classical formulations, DMEM plus 10% fetal bovine serum, and tri-lineage differentiation of cells into adipocytes, chondrocytes, and osteoblasts.

### Example 7

### Neural Stem Cell (NSC) Adherent Growth in Stempro® NSC SFM

7.1 Procedure

CELLstart™ with 0.92 mg/ml fibronectin and 20 mg/ml of HSA is diluted 1:50 to 1:100 in Dulbecco's Phosphate Buffered Saline. A sufficient volume of dilute CELLstart™ solution was added to coat tissue culture containers, i.e., 10 ml for T75 flask, 2.5 ml for a 60 mm dish, and 1.5 ml for a 35 mm dish. The culture containers were then incubated in a 37° C., 5% CO₂ incubator for 60 to 120 minutes.

After incubation, neural stem cells were seeded onto the culture containers at a density of 0.1 to 1×10⁵ cells/cm². Cells were passaged into the serum-free media, StemPro® NSC SFM (Invitrogen, Carlsbad, Calif., US), when they are ˜90% confluent.

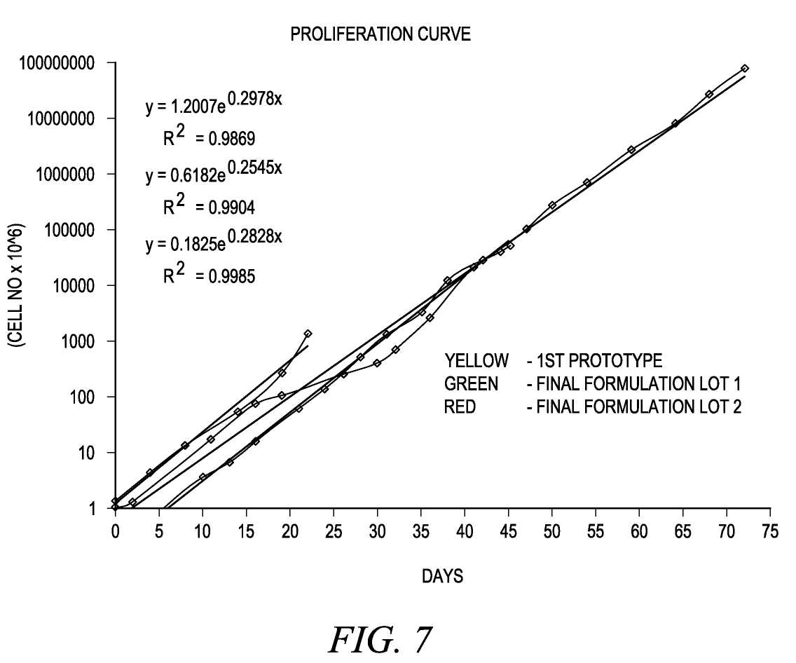

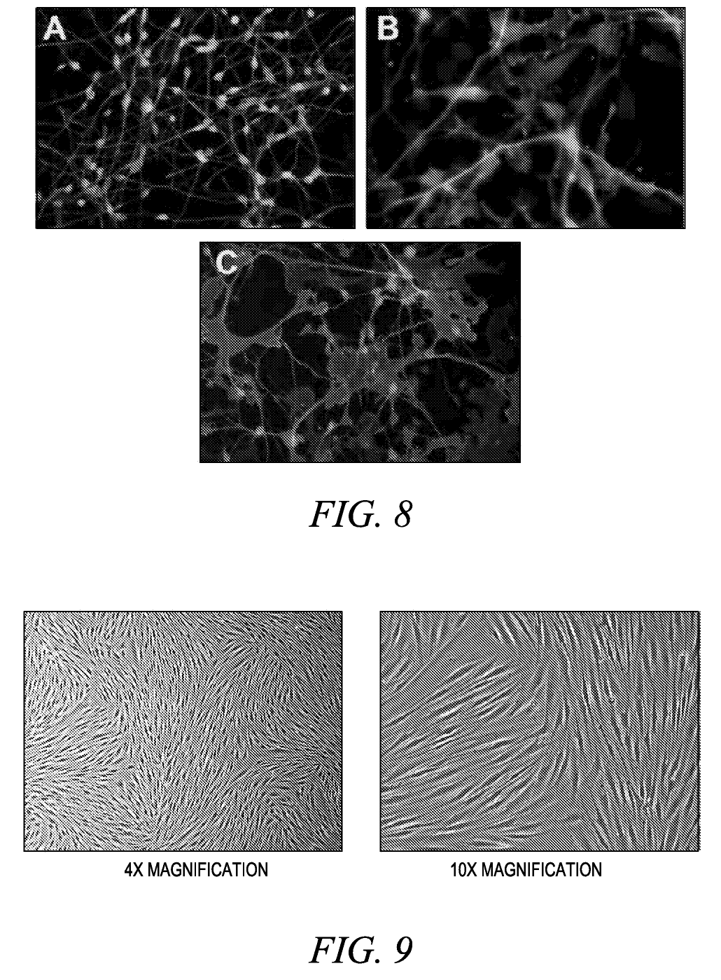

Neural stem cells cultured on CELLstart™ coated culture containers in StemPro® NSC SFM were able to maintain their differentiation potential. Cells were differentiated to make neurons and glial cells. FIG. 7 shows the growth curve of neural stem cells cultured in StemPro® NSC SFM. FIG. 8 shows the differentiation of neural stem cells cultured in StemPro® NSC SFM, with panel A showing differentiation into neurons, and panels B and C showing differentiation into glial cells. A) Neurons were labeled with HuC/D(green) and Dcx(red). B) Cells whose lineage to oligodendrocytes were labeled with GalC(red). Cell nuclei were labeled with Dapi(blue) and neurons were labeled with Dcx(green). C) Cells whose lineage to Astrocytes were labeled with CD44(green). Cell nuclei were labeled with Dapi(blue) and neurons were labeled with Dcx (green).

7.2 Conclusion

Neural stem cells cultured in CELLstart™ coated culture containers and StemPro® NSC SFM were able to grow, expand, and maintain differentiation potential as evidence by supporting growth and differentiation data.

### Example 8

### Stem Cell Culture with Knockout™ SR Xenofree Supplemented Media (Shelf-Life Studies)

8.1 Procedure

BG01v cells were grown in a series of duplicate (test and control) 35 mm dishes coated with a 1:50 dilution of CELLstart™ (made according to Example 1) and DMEM/F12 supplemented with Knockout™ SR XenoFree (Gibco/Invitrogen, Carlsbad, Calif., US). The BG01v cells were from frozen cells which had been through 15 passages on CELLstart™ using Knockout SR™ XenoFree supplemented media.

Two CELLstart™ solutions were compared, one which had been stored for 19 months at about 2° C. to about 8° C. (test) and the other had been made for use as a control for this study (control). A total of 8 dishes were pre-coated for the test and control CELLstart™ samples. Two dishes from test and control samples were used for the assay setup and the other 6 dishes were parafilmed and stored at 2° C. to 8° C. to use for passaging cells in this assay.

Cells were passaged 3 times in test and control CELLstart™ samples. Cells were passaged every 3 to 4 days when cells reach around 90% confluency, with detachment of the cells being accomplished using TrypLE™ Select (Gibco/Invitrogen, Carlsbad, Calif., US).

Conclusions

It can be concluded that CELLstart™ at 19 months will support BG01v cell attachment, growth, and undifferentiated expansion when used at 1:50 dilution to pre-coat tissue culture dishes. The undifferentiated cell morphology in dishes pre-coated with the 19 month sample of CELLstart™ were found to be comparable to the control lot of CELLstart™ for all 3 passages.

### Example 9

### Human Fetal Fibroblasts Cultured on Cellstart™ Coated Flasks

9.1 Procedure

Human fetal fibroblasts (HFF) were cultured on 1:50 CELLstart coated T-flasks (prepared according to Example 1) in Knockout™ DMEM supplemented with 15% Knockout SR XenoFree (Gibco/Invitrogen, Carlsbad, Calif., US), 4 mM GlutaMAX, 0.1 mM MEM non-essential amino acids, and 0.1 mM β-mercaptoethanol. Cells were cultured and fluid changed every 3-4 days.

Confluent cells were passaged, cryopreserved, or used as feeders for the growth of human embryonic stem cells when cell density reaches ˜90% confluency.

FIG. 9 depicts HFF cultured under conditions described in the 9.1 PROCEDURE above.

Conclusion

It can be concluded based on studies performed (FIG. 9) that CELLstart™ will support the attachment and healthy expansion of human fetal fibroblasts when cultured under serum-free and xeno-free conditions.

What is claimed is:

1. A composition of matter for coating a substrate with a cell matrix, wherein:
(a) the cell matrix consists essentially of:
(i) Fibronectin, and
(ii) Albumin;
(b) the Fibronectin and said Albumin are present at concentrations effective for forming a cellular matrix when contacted with the substrate; and
(c) the cell matrix supports the growth of cells in the presence of culture media.

2. The composition of matter of claim 1, wherein the composition of matter is serum-free.

3. The composition of matter of claim 1, wherein the substrate is a surface on a culture vessel.

4. The composition of matter of claim 1, wherein the cells are stem cells.

5. The composition of matter of claim 1, wherein the cells are primary cells.

6. The composition of matter of claim 1, wherein the Fibronectin is plasma fibronectin.

7. The composition of matter of claim 1, wherein the Albumin is serum albumin.

8. A composition of matter comprising a substrate coated with a cell matrix and a cell in adherence the cell matrix, wherein:
(a) the cell matrix consists essentially of Fibronectin and Albumin, where the Fibronectin and the Albumin are present at concentrations effective for forming a cellular matrix when contacted with the substrate, and the cell matrix supports the growth of cells in the presence of culture media; and
(b) the cell is a stem cell or a primary cell.

9. The composition of matter of claim 8, wherein the cell matrix is serum-free.

10. The composition of claim 8, wherein the stem cell is an embryonic stem cell.

11. The composition of matter of claim 8, wherein the primary cell is selected from the group consisting of a keratinocyte cell and fibroblast cell.

12. The composition of matter of claim 8, wherein the Fibronectin is plasma fibronectin.

13. The composition of matter of claim 8, wherein the Albumin is serum albumin.

14. An article of manufacture comprising a surface coated with a stem cell matrix, wherein the stem cell matrix consists essentially of Fibronectin and Albumin at concentrations effective for forming a cellular matrix.

15. The article of manufacture of claim 14, wherein the stem cell matrix is serum-free.

16. The article of manufacture of claim 14, wherein the article of manufacture of matter is serum-free.

17. A stem cell culture, comprising:
(a) a population of stem cells;
(b) a culture medium; and
(c) a stem cell matrix, wherein:
(1) the stem cell matrix consists essentially of Fibronectin and Albumin;
(2) the Fibronectin and the Albumin are present at concentrations effective for forming a cellular matrix when contacted with a substrate; and
(3) the stem cell matrix supports the growth of stem cells in the presence of culture media.

18. The stem cell culture of claim 17, wherein the stem cell culture is serum-free.

19. The stem cell culture of claim 17, wherein the stem cells are embryonic stem cells.

20. The stem cell culture of claim 17, wherein the Fibronectin is plasma fibronectin.

21. The stem cell culture of claim 17, wherein the Albumin is serum albumin.

22. An article of manufacture comprising a stem cell culture, the stem cell culture comprising:
(a) a population of stem cells;
(b) a culture medium; and
(c) a stem cell matrix, wherein:
(i) the stem cell matrix consists essentially of:
1. Fibronectin, and
2. Albumin;
(ii) the Fibronectin and the Albumin are present at concentrations effective for forming a cellular matrix when contacted with a substrate; and
(iii) the stem cell matrix supports the growth of stem cells in the presence of culture media.

23. The article of manufacture of claim 22, wherein the stem cell culture is serum-free.

24. The article of manufacture of claim 22, wherein the stem cells are embryonic stem cells.

25. The article of manufacture of claim 22, wherein the Fibronectin is plasma fibronectin.

26. The article of manufacture of claim 22, wherein the Albumin is serum albumin.

27. A method of preparing an article of manufacture comprising a surface coated with a stem cell matrix, wherein the stem cell matrix consists essentially of Fibronectin and Albumin at concentrations effective for forming a cellular matrix, the method comprising the following steps, carried out simultaneously or in any order:
(a) coating a substrate surface of the article of manufacture with Fibronectin; and
(b) coating an overlapping portion of the substrate surface of the article of manufacture with Albumin.

28. The method of claim 27, wherein steps (a) and (b) are carried out simultaneously.

29. The method of claim 27, wherein the stem cell matrix is serum-free.

30. The method of claim 27, wherein the article of manufacture is serum-free.

31. The method of claim 27, wherein the Fibronectin is human plasma fibronectin (HPFN), and the Albumin is human serum albumin (HSA).

32. A method of generating a stem cell culture, comprising the following steps:
(a) combining in a culture vessel, simultaneously or in any order:
(i) a stem cell matrix, wherein the stem cell matrix consists essentially of Fibronectin and Albumin,
(ii) a culture medium, and
(iii) stem cells; and
(b) incubating the culture vessel.

33. The method of claim 32, wherein the stem cell culture is serum-free.

34. The method of claim 32, wherein the stem cells are embryonic stem cells.

35. The method of claim 32, wherein the Fibronectin is plasma fibronectin.

36. The method of claim 31, wherein the Albumin is serum albumin.

37. A kit comprising a carrier means compartmentalized to receive in close confinement therein one or more container means, wherein a first container means contains a matrix-forming formulation, wherein:
(a) the matrix-forming formulation consists essentially of:
(i) Fibronectin, and
(ii) Albumin;
(b) the Fibronectin and the Albumin are present at concentrations effective for forming a cellular matrix when contacted with a substrate; and
(c) the cell matrix supports the growth of primary cells or stem cells in the presence of culture media.

38. The kit of claim 37, wherein the kit further comprises one or more additional container means.

39. The kit of claim 38, wherein the one or more additional container means comprises a formulation.

40. The kit of claim 39, wherein the formulation is selected from the group consisting of:
(a) a culture medium;
(b) an energy source;
(c) a buffering agent;
(d) a salt; and
(e) a composition comprising an antibody.

41. The kit of claim 39, wherein the formulation is a composition comprising an antibody, and the antibody is specific for an antigen selected from the group consisting of:
(a) SSEA-3;
(b) SSEA-4;
(c) TRA-1-60; and
(d) TRA-1-81.

42. A kit comprising a carrier means compartmentalized to receive in close confinement therein one or more container means, wherein a first container means contains a stem cell matrix, wherein:
(a) the stem cell matrix consists essentially of:
(i) Fibronectin, and
(ii) Albumin;
(b) the Fibronectin and the Albumin are present at concentrations effective for forming a cellular matrix when contacted with a substrate; and
(c) the stem cell matrix supports the growth of stem cells in the presence of culture media.

43. A kit comprising a carrier means compartmentalized to receive in close confinement therein one or more container means, wherein a first container means contains a stem cell culture, wherein the stem cell culture comprises:
(a) a population of stem cells;
(b) a culture medium; and
(c) a stem cell matrix, wherein:
(1) the stem cell matrix consists essentially of:
(i) Fibronectin and
(ii) Albumin;
(2) the Fibronectin and the Albumin are present at concentrations effective for forming a cellular matrix when contacted with a substrate; and
(3) the stem cell matrix supports the growth of stem cells in the presence of culture media.
